SEQUENCE LISTING (88 sequences)

SEQ ID NO: 1 - protein, 22 aa, Pseudomonas species
QRLSTGSRINSAKDDAAGLQIA

SEQ ID NO: 2 - protein, 18 aa, Pseudomonas species
SKEKFERTKPHVNVGTIG

SEQ ID NO: 3 - DNA, 23 bp, synthetic construct
  source 1..23: mol_type = other DNA
tccctcagcacattccagcagat

SEQ ID NO: 4 - DNA, 24 bp, synthetic construct
  source 1..24: mol_type = other DNA
aacgattcctggacctgcctcatc

SEQ ID NO: 5 - DNA, 20 bp, synthetic construct
  source 1..20: mol_type = other DNA
acggggaaaacttagcctgg

SEQ ID NO: 6 - DNA, 20 bp, synthetic construct
  source 1..20: mol_type = other DNA
ttggcacatccgagtctcac

SEQ ID NO: 7 - DNA, 22 bp, synthetic construct
  source 1..22: mol_type = other DNA
ggttggaatatggtagtgctgt

SEQ ID NO: 8 - DNA, 20 bp, synthetic construct
  source 1..20: mol_type = other DNA
ctgaacggatgaaggtggaa

SEQ ID NO: 9 - DNA, 20 bp, synthetic construct
  source 1..20: mol_type = other DNA
caacttcccacgtcagccct

SEQ ID NO: 10 - DNA, 20 bp, synthetic construct
  source 1..20: mol_type = other DNA
cggggctgctgcacttctaa

SEQ ID NO: 11 - DNA, 22 bp, synthetic construct
  source 1..22: mol_type = other DNA
cggagatggagacaagtggctt

SEQ ID NO: 12 - DNA, 24 bp, synthetic construct
  source 1..24: mol_type = other DNA
tgtgaggatcgtttgtgtggagaa

SEQ ID NO: 13 - DNA, 20 bp, synthetic construct
  source 1..20: mol_type = other DNA
ccctttggaatccccgtttg

SEQ ID NO: 14 - DNA, 24 bp, synthetic construct
  source 1..24: mol_type = other DNA
gaatagaacccaccaaatcctcca

SEQ ID NO: 15 - DNA, 20 bp, synthetic construct
  source 1..20: mol_type = other DNA
acaccaccattagcctcgcc

SEQ ID NO: 16 - DNA, 20 bp, synthetic construct
  source 1..20: mol_type = other DNA
acgcggggaaagttgtgtca

SEQ ID NO: 17 - DNA, 20 bp, synthetic construct
  source 1..20: mol_type = other DNA
acggtggcgatcaaaaacgc

SEQ ID NO: 18 - DNA, 20 bp, synthetic construct
  source 1..20: mol_type = other DNA
tgggacaaggatgcgacgac

SEQ ID NO: 19 - DNA, 20 bp, synthetic construct
  source 1..20: mol_type = other DNA
gctggtgcacagaaagcgtc

SEQ ID NO: 20 - DNA, 20 bp, synthetic construct
  source 1..20: mol_type = other DNA
cctcaaggcgtaccctgcaa

SEQ ID NO: 21 - DNA, 21 bp, synthetic construct
  source 1..21: mol_type = other DNA
acctcttcgaatctccccatc

SEQ ID NO: 22 - DNA, 21 bp, synthetic construct
  source 1..21: mol_type = other DNA
aacttcattgatcggcgtcac

SEQ ID NO: 23 - DNA, 26 bp, synthetic construct
  source 1..26: mol_type = other DNA
ctacaagtgtttccgttgactctgaa

SEQ ID NO: 24 - DNA, 27 bp, synthetic construct
  source 1..27: mol_type = other DNA
acgaaattatcgcagtacattagagga

SEQ ID NO: 25 - DNA, 24 bp, synthetic construct
  source 1..24: mol_type = other DNA
cacgactgtcgataattttgggtt

SEQ ID NO: 26 - DNA, 24 bp, synthetic construct
  source 1..24: mol_type = other DNA
catattgtcatcttcgttccagga

SEQ ID NO: 27 - DNA, 25 bp, synthetic construct
  source 1..25: mol_type = other DNA
aacaaaaagaaaccgtatctgccac

SEQ ID NO: 28 - DNA, 24 bp, synthetic construct
  source 1..24: mol_type = other DNA
tcctaacttcacgcatgctatctc

SEQ ID NO: 29 - DNA, 22 bp, synthetic construct
  source 1..22: mol_type = other DNA
ctccttcttccttgcaaatgga

SEQ ID NO: 30 - DNA, 23 bp, synthetic construct
  source 1..23: mol_type = other DNA
gagaatcatcaagaagcaaaggg

SEQ ID NO: 31 - DNA, 24 bp, synthetic construct
  source 1..24: mol_type = other DNA
ggcaacaaaccatgtcgtatcaag

SEQ ID NO: 32 - DNA, 25 bp, synthetic construct
  source 1..25: mol_type = other DNA
cgttgacactcttcttctctaaccg

SEQ ID NO: 33 - DNA, 24 bp, synthetic construct
  source 1..24: mol_type = other DNA
aatctgaactacatcccattcgga

SEQ ID NO: 34 - DNA, 24 bp, synthetic construct
  source 1..24: mol_type = other DNA
ttagatcaggttgatccccatttg

SEQ ID NO: 35 - DNA, 26 bp, synthetic construct
  source 1..26: mol_type = other DNA
cgtttggatcgtgtaggagaatatgt

SEQ ID NO: 36 - DNA, 21 bp, synthetic construct
  source 1..21: mol_type = other DNA
tgttccccattgatgtctttg

SEQ ID NO: 37 - DNA, 23 bp, synthetic construct
  source 1..23: mol_type = other DNA
ctggaatgctgttgtctctgttg

SEQ ID NO: 38 - DNA, 23 bp, synthetic construct
  source 1..23: mol_type = other DNA
tcgccttttgatttcttcccagt

SEQ ID NO: 39 - DNA, 20 bp, synthetic construct
  source 1..20: mol_type = other DNA
gacacccgaaaggcgaaagg

SEQ ID NO: 40 - DNA, 20 bp, synthetic construct
  source 1..20: mol_type = other DNA
cagcgagagcagcatcaagc

SEQ ID NO: 41 - DNA, 20 bp, synthetic construct
  source 1..20: mol_type = other DNA
gtcggtcacgcgctcaggta

SEQ ID NO: 42 - DNA, 21 bp, synthetic construct
  source 1..21: mol_type = other DNA
cgagatgctgacatggccaga

SEQ ID NO: 43 - DNA, 22 bp, synthetic construct
  misc_feature 1..22: note = primer sequence
  source 1..22: mol_type = other DNA
ttggggcgtggaatggagacag

SEQ ID NO: 44 - DNA, 25 bp, synthetic construct
  misc_feature 1..25: note = primer sequence
  source 1..25: mol_type = other DNA
catagtggttacagagtgttgagcc

SEQ ID NO: 45 - DNA, 22 bp, synthetic construct
  misc_feature 1..22: note = primer sequence
  source 1..22: mol_type = other DNA
gaagtcacttggtcgtcggttg

SEQ ID NO: 46 - DNA, 22 bp, synthetic construct
  misc_feature 1..22: note = primer sequence
  source 1..22: mol_type = other DNA
tgcttcttcttcatgtacccat

SEQ ID NO: 47 - DNA, 20 bp, synthetic construct
  misc_feature 1..20: note = primer sequence
  source 1..20: mol_type = other DNA
gaaaaggaagggatccttgg

SEQ ID NO: 48 - DNA, 20 bp, synthetic construct
  misc_feature 1..20: note = primer sequence
  source 1..20: mol_type = other DNA
gcaaagcattgactgtgctc

SEQ ID NO: 49 - DNA, 25 bp, synthetic construct
  misc_feature 1..25: note = primer sequence
  source 1..25: mol_type = other DNA
cttggttcagtatgacaaaccttac

SEQ ID NO: 50 - DNA, 25 bp, synthetic construct
  misc_feature 1..25: note = primer sequence
  source 1..25: mol_type = other DNA
gaccaggtctagcaactaaagaagt

SEQ ID NO: 51 - DNA, 22 bp, synthetic construct
  misc_feature 1..22: note = primer sequence
  source 1..22: mol_type = other DNA
tcggtttgtgtagtgtcagagc

SEQ ID NO: 52 - DNA, 20 bp, synthetic construct
  misc_feature 1..20: note = primer sequence
  source 1..20: mol_type = other DNA
aaggtcaaaccagcaggacc

SEQ ID NO: 53 - DNA, 22 bp, synthetic construct
  misc_feature 1..22: note = primer sequence
  source 1..22: mol_type = other DNA
ggacatgaaacatcggcggcag

SEQ ID NO: 54 - DNA, 22 bp, synthetic construct
  misc_feature 1..22: note = primer sequence
  source 1..22: mol_type = other DNA
gaggagctcctggtgcaatctg

SEQ ID NO: 55 - DNA, 22 bp, synthetic construct
  misc_feature 1..22: note = primer sequence
  source 1..22: mol_type = other DNA
gcacggaagcttcgaaaagagg

SEQ ID NO: 56 - DNA, 21 bp, synthetic construct
  misc_feature 1..21: note = primer sequence
  source 1..21: mol_type = other DNA
ccgaggagatcagcgacatcg

SEQ ID NO: 57 - DNA, 22 bp, synthetic construct
  misc_feature 1..22: note = primer sequence
  source 1..22: mol_type = other DNA
ctgtgcttggtcaaggtccacc

SEQ ID NO: 58 - DNA, 20 bp, synthetic construct
  misc_feature 1..20: note = primer sequence
  source 1..20: mol_type = other DNA
agatggatcaaagccagccc

SEQ ID NO: 59 - DNA, 21 bp, synthetic construct
  misc_feature 1..21: note = primer sequence
  source 1..21: mol_type = other DNA
cttggcaaatggagggaggtg

SEQ ID NO: 60 - DNA, 19 bp, synthetic construct
  misc_feature 1..19: note = primer sequence
  source 1..19: mol_type = other DNA
cgttatgcctaacccggcg

SEQ ID NO: 61 - DNA, 23 bp, synthetic construct
  misc_feature 1..23: note = primer sequence
  source 1..23: mol_type = other DNA
gttgggatggagttttgggcaag

SEQ ID NO: 62 - DNA, 22 bp, synthetic construct
  misc_feature 1..22: note = primer sequence
  source 1..22: mol_type = other DNA
ctgtgtggtgtagctggtgagc

SEQ ID NO: 63 - DNA, 23 bp, synthetic construct
  misc_feature 1..23: note = primer sequence
  source 1..23: mol_type = other DNA
ccatgcctcagcttcttactgag

SEQ ID NO: 64 - DNA, 21 bp, synthetic construct
  misc_feature 1..21: note = primer sequence
  source 1..21: mol_type = other DNA
gtacaagctccctgtgctggc

SEQ ID NO: 65 - DNA, 20 bp, synthetic construct
  misc_feature 1..20: note = primer sequence
  source 1..20: mol_type = other DNA
tgcaatggcgaggatggctg

SEQ ID NO: 66 - DNA, 23 bp, synthetic construct
  misc_feature 1..23: note = primer sequence
  source 1..23: mol_type = other DNA
atgcatcttcgaaacctgagacg

SEQ ID NO: 67 - DNA, 21 bp, synthetic construct
  misc_feature 1..21: note = primer sequence
  source 1..21: mol_type = other DNA
tgccagctgcatgagttccac

SEQ ID NO: 68 - DNA, 21 bp, synthetic construct
  misc_feature 1..21: note = primer sequence
  source 1..21: mol_type = other DNA
aattctctccacgagcgcagg

SEQ ID NO: 69 - DNA, 21 bp, synthetic construct
  misc_feature 1..21: note = primer sequence
  source 1..21: mol_type = other DNA
tccggtgtacgagcttcatgg

SEQ ID NO: 70 - DNA, 18 bp, synthetic construct
  misc_feature 1..18: note = primer sequence
  source 1..18: mol_type = other DNA
ccagacaactctggcaac

SEQ ID NO: 71 - DNA, 20 bp, synthetic construct
  misc_feature 1..20: note = primer sequence
  source 1..20: mol_type = other DNA
ccgagccgtggcctaaagtc

SEQ ID NO: 72 - DNA, 21 bp, synthetic construct
  misc_feature 1..21: note = primer sequence
  source 1..21: mol_type = other DNA
cacgacctgcttcgccaaatc

SEQ ID NO: 73 - DNA, 20 bp, synthetic construct
  misc_feature 1..20: note = primer sequence
  source 1..20: mol_type = other DNA
taggagtgttgcagcggagc

SEQ ID NO: 74 - DNA, 21 bp, synthetic construct
  misc_feature 1..21: note = primer sequence
  source 1..21: mol_type = other DNA
atcaaccgtcagttccacccc

SEQ ID NO: 75 - DNA, 21 bp, synthetic construct
  misc_feature 1..21: note = primer sequence
  source 1..21: mol_type = other DNA
tagcacgacatccgagagcag

SEQ ID NO: 76 - DNA, 20 bp, synthetic construct
  misc_feature 1..20: note = primer sequence
  source 1..20: mol_type = other DNA
aaggtccacttccacctcgc

SEQ ID NO: 77 - DNA, 22 bp, synthetic construct
  misc_feature 1..22: note = primer sequence
  source 1..22: mol_type = other DNA
gagcttcgtggaggtgtcaaag

SEQ ID NO: 78 - DNA, 20 bp, synthetic construct
  misc_feature 1..20: note = primer sequence
  source 1..20: mol_type = other DNA
ggttcatggtgagtgggagg

SEQ ID NO: 79 - DNA, 22 bp, synthetic construct
  misc_feature 1..22: note = primer sequence
  source 1..22: mol_type = other DNA
ccgcatctgcggttgatttccc

SEQ ID NO: 80 - DNA, 20 bp, synthetic construct
  misc_feature 1..20: note = primer sequence
  source 1..20: mol_type = other DNA
ccatatgggaccctgtgcgg

SEQ ID NO: 81 - DNA, 20 bp, synthetic construct
  misc_feature 1..20: note = primer sequence
  source 1..20: mol_type = other DNA
agtacgccggagatgagtcg

SEQ ID NO: 82 - DNA, 20 bp, synthetic construct
  misc_feature 1..20: note = primer sequence
  source 1..20: mol_type = other DNA
cgaaaaacggttctccgccg

SEQ ID NO: 83 - DNA, 23 bp, synthetic construct
  misc_feature 1..23: note = primer sequence
  source 1..23: mol_type = other DNA
cctctaaacgggtggaacaagcc

SEQ ID NO: 84 - DNA, 22 bp, synthetic construct
  misc_feature 1..22: note = primer sequence
  source 1..22: mol_type = other DNA
cctcggaacctctaacctcgac

SEQ ID NO: 85 - DNA, 20 bp, synthetic construct
  misc_feature 1..20: note = primer sequence
  source 1..20: mol_type = other DNA
ggctagcgtggaggttccag

SEQ ID NO: 86 - DNA, 25 bp, synthetic construct
  misc_feature 1..25: note = primer sequence
  source 1..25: mol_type = other DNA
ccacaatatcaaccttggagagagc

SEQ ID NO: 87 - DNA, 22 bp, synthetic construct
  misc_feature 1..22: note = primer sequence
  source 1..22: mol_type = other DNA
attgacgggtttgggaaccgtg

SEQ ID NO: 88 - DNA, 20 bp, synthetic construct
  misc_feature 1..20: note = primer sequence
  source 1..20: mol_type = other DNA
caggctcgtggaccgatacc

(19) United States
(12) Patent Application Publication
(10) Pub. No.: US 2024/0344991 A1
(43) Pub. Date: Oct. 17, 2024

(54) REAL TIME DETECTION AND QUANTITATION OF PLANT INNATE IMMUNITY RESPONSE USING RAMAN SPECTROSCOPY

(71) Applicants: TEMASEK LIFE SCIENCES LABORATORY LIMITED, Singapore (SG); MASSACHUSETTS INSTITUTE OF TECHNOLOGY, Cambridge, MA (US)

(72) Inventors: Pil Joong CHUNG, Singapore (SG); Sayuj KOYYAPPURATH, Singapore (SG); Nam-Hai CHUA, Singapore (SG); Rajani SAROJAM, Singapore (SG); Gajendra Pratap SINGH, Singapore (SG); Rajeev J. RAM, Cambridge, MA (US)

(73) Assignees: TEMASEK LIFE SCIENCES LABORATORY LIMITED, Singapore (SG); MASSACHUSETTS INSTITUTE OF TECHNOLOGY, Cambridge, MA (US)

(21) Appl. No.: 18/685,153

(22) PCT Filed: Aug. 19, 2022
(86) PCT No.: PCT/SG2022/050593

§ 371 Date: Feb. 20, 2024

(30) Foreign Application Priority Data
Aug. 20, 2021 (SG) 10202109110S

(51) Int. Cl. G01N 21/65; G01N 33/00

(52) U.S. Cl. CPC G01N 21/65; G01N 33/0098

(57) ABSTRACT

The present invention relates to the use of Raman spectroscopy for the real time detection and quantitation of innate immunity response in plants. More specifically, the present invention provides Raman spectroscopy as a tool for rapid, non-invasive, and early detection and quantitation of plant innate immune response.

### FIELD

The present invention relates to the use of Raman spectroscopy for the real time detection and quantitation of innate immunity response in plants. More specifically, the present invention provides Raman spectroscopy as a tool for rapid, non-invasive, and early detection and quantitation of plant innate immune response.

### BACKGROUND

The publications and other materials used herein to illuminate the background of the invention or provide additional details respecting the practice, are incorporated by reference, and for convenience are respectively grouped in the Bibliography.

Global food supply and security have been challenged by water scarcity and climate change and the situation is further exacerbated by the projected increase in the world's population to about 10 billion by 2050 (Misra, 2014). The expected increase in food demand requires a corresponding increase in crop productivity and disruptive improvements in agricultural production systems. Crop productivity in the field is compromised by abiotic stresses as well as by biotic stresses and the potential agricultural yield losses caused by plant pathogens are about 16% globally (Oerke, 2006).

Several strategies have been implemented to mitigate the degradation of crop yield caused by plant diseases. The generation of pathogen-resistant plants by transgenic means (Dong & Ronald, 2019; van Esse et al., 2020) and the use of agrochemicals to confer disease resistance (Zhou & Wang, 2018) have met with some success. In the field, rapid detection of pathogen-infected plants is an important first step in plant disease management. Early detection of infected plants would allow their rapid and selective removal thus greatly reducing pathogen load and the opportunity for further disease spreading. Visual inspection of disease symptoms and grading of their disease severity is a classical method of identifying infected plants. However, most disease symptoms are usually manifest only at relatively late stages of infection when the pathogen load is already quite high. For this reason, over the last two decades, plant disease diagnosis has shifted from visual inspection of symptom-based disease phenotype to molecular-based diagnostic procedures. Compared to the visual method of plant disease diagnosis, current molecular methods such as polymerase chain reaction (PCR), fluorescence in-situ hybridization (FISH), immunofluorescence (IF), and gas chromatography-mass spectrometry (GC-MS) are widely available and these methods offer greater sensitivity, accuracy, and precision for pathogen detection (Fang & Ramasamy, 2015; Martinelli et al., 2015). Nevertheless, these methods suffer from several drawbacks: (1) they depend on the availability of pathogen gene sequences or specific antibodies to pathogen proteins; (2) the assays are not amenable to on-site field application as the diseased samples have to be collected and analyzed in the laboratory; (3) the assays are time-consuming and laborious to perform and in some case require the use of reagents that may be toxic.

To obviate the drawbacks of molecular methods, several groups have explored the use of Raman spectroscopy for rapid diagnosis of plant diseases (Yeturu et al., 2016; Egging et al., 2018; Farber & Kurouski, 2018; Farber et al., 2019; Mandrile et al., 2019; Faber et al., 2020a & b; Sanchez et al., 2020b). A Raman spectrum records the molecular vibrations of cellular metabolites without the use of a label or reagents. Differences in the Raman spectra of a diseased sample versus that of the control sample are fingerprints reflecting changes in cellular metabolites following pathogen infection. Acquisition of a Raman spectrum usually takes only less than a minute and with a hand-held device such recoding can be directly done on field crops (Gupta et al., 2020).

Raman spectroscopy-based detection and identification of bacterial, fungal and viral diseases in plants have been reported. (Yeturu et al., 2016, Farber & Kurouski, 2018, Mandrile et al., 2019, Farber et al., 2019, Sanchez et al., 2020 b,c, Payne and Kurouski, 2021) While Raman fingerprints vary depending on the disease symptoms and the pathogens involved, in most cases, the Raman spectra collected from diseased leaves primarily show changes in peak intensities corresponding to vibrational bands originating from carotenoids, pectin, phenylpropanoids, cellulose and proteins. In most cases, irrespective of the responsible pathogen, a decrease in carotenoid (vibrational bands around 1,523 cm⁻¹ and 1,541 cm⁻¹) was consistently seen in the symptomatic compared to the asymptomatic tissues. Using Raman spectroscopy Rys et al. (2014) reported an increase in carotenoid bands during Obuda pepper virus infection at 48 hpi; however, measurement by traditional spectrophotometric methods did not show significant changes in total carotenoid content. This discrepancy was attributed to a qualitative rather than quantitative change in total carotenoids or to the ability of Raman spectroscopy to detect different sets of carotenoids in comparison to spectrophotometric method. Raman spectroscopy has been used to identify Arabidopsis plants suffering from the early stages of nitrogen deficiency (Huang et al., 2020). In rice, a hand-held Raman spectrometer showed a highly accurate pre-symptomatic diagnostics of N, P, and K deficiencies as well as a medium and high salinity stresses with hydroponic condition (Sanchez et al., 2020a).

Plants have evolved a multi-layer defense system to combat offending pathogens (Li & Staiger, 2018). A major defense system is the innate immunity pathway based on the specific recognition of pathogen-derived molecules by hosts (Ausubel, 2005; Abdul et al., 2020). Plants have developed transmembrane receptors called pattern recognition receptors (PRRs) that specifically recognize pathogen-derived molecules in the apoplastic space. Upon binding to these molecules carrying pathogen-associated molecular patterns (PAMP) the PRRs initiate signaling of the innate immune pathway referred to as PAMP-triggered immunity (PTI). PTI causes rapid plant defence responses, including calcium ion flux, ROS burst, activation of the MAPK cascades, expression of defence-related genes and callose deposition. (Jones & Dangl, 2006; Luna et al., 2011; Bigeard et al., 2015). Two of the most studied PRRs are FLS2 (flagellin-sensitive 2) which senses a conserved 22-amino acid sequence of flagellin (flg22) and EFR (EF-Tu receptor) which recognizes a conserved 18-amino acid peptide (elf18) of the elongation factor Tu (EF-Tu) (Zipfel et al., 2004; Chinchilla et al., 2006; Zipfel et al., 2006). The signaling mechanisms of these two PRRs have been recently reviewed (Peng et al., 2018; Saijo et al., 2018). Briefly, recognition of PAMPs by PRRs induces their association with the co-receptor BAK1 (BRASSINOSTEROID INSENSITIVE 1-associated receptor kinase 1) resulting in the phosphorylation of both receptor kinases (Sun et al., 2013). The PRRs-BAK1 complex directly phosphorylates the cytosolic BIK1 (BOTRYTIS-INDUCED KINASE1) (Halter et al., 2014), which in turn activates RBOHD (Respiratory Burst Oxidase Homologue D; NADPH oxidase) by phosphorylation of its N-terminal regulatory domain elevating production of reactive oxygen species (ROSs) and subsequent PAMP-triggered immunity (PTI) (Yu et al., 2017). On the other hand, PUB12 and PUB13, which are ubiquitin E3 ligases, form a BAK1-dependent complex with FLS2 and are able to polyubiquitinate FLS2 (Lu et al., 2011) and induce receptor endocytosis and subsequent degradation (Robatzek et al., 2006). Recent work showed that the cytosolic kinase BIK1 is down-regulated by PUB25/26 which mediates its ubiquitination and degradation by 26S proteasomes (Wang et al., 2018).

It is desired to develop a non-invasive method for the diagnosis of plants that exhibit early host responses to pathogen infection.

### SUMMARY OF THE INVENTION

The present invention relates to the use of Raman spectroscopy for the real time detection and quantitation of innate immunity response in plants. More specifically, the present invention provides Raman spectroscopy as a tool for rapid, non-invasive, and early detection and quantitation of plant innate immune response.

To demonstrate the use of Raman spectroscopy for detecting early changes in cellular metabolites following pathogen-plant interaction, the elicitors flg22 and elf18 were used to trigger PTI in Arabidopsis plants. Raman spectra were taken in the leaf region proximal to the mock-infiltrated site or the elicitor infiltrated site and the difference Raman spectrum revealed several peaks along with carotenoids that showed significant changes at 24 hpi. To quantify the early PTI response using Raman spectroscopy, an elicitor response factor (ERF) was developed whereby a higher ERF value indicates a more significant elicitor-induced immune response. Investigations of several PTI-related mutants produced the expected results substantiating the reliability and reproducibility of this method. This approach was further validated with pathogen studies in Arabidopsis and the vegetable crop-Choysum (Brassica rapa v. parachinenesis). The non-invasive optical method described here can be used to identify early infection of plants, including Arabidopsis and field crops.

The invention uses Raman spectral signature of plant metabolites as biomarkers for an early, real-time diagnosis of pathogen infection in growing plants in a non-invasive or non-destructive way, wherein plants need not be “destroyed” in order to detect pathogen infection. Raman spectroscopy at near-infrared (830 nm) excitation wavelength accurately detects changes in concentration of different plant metabolites due to plant infection by changes in intensity of Raman signals at various wavenumbers (Tables 2 to 5) of which, changes at 1001 cm⁻¹, 1151 cm⁻¹ and 1521 cm⁻¹ for carotenoids and 1550 cm⁻¹ for proteins are the most significant. Transient increases (in plant metabolites, particularly carotenoids and proteins are detected before manifestation of any disease symptoms, highlighting carotenoids and proteins as indicator metabolites for identifying early onset of infection, e.g., at about 10 hours to about 24 hours after receptor activation, and Raman spectroscopy as a predictive tool for early diagnosis. Raman-based signatures can be used in a hand-held Raman spectroscope to detect the early onset of infection in plants. Any Raman spectroscope can be used in the invention for detecting early infection in plants.

The early, real-time diagnosis of early infection in plants, e.g., in individual plants in a plant population, enables the physical removal of early stage infected plants and thus contain the spread of infection. Containing the spread of infection will ensure yield of plants, such as leafy vegetables growing in, particularly, artificial urban farming settings.

Thus, in one aspect, the present invention provides a method of diagnosing early onset of pathogen infection of a plant. In accordance with this aspect, the method comprises:
(a) obtaining a Raman spectra of carotenoids and/or proteins in vivo and in situ (i.e., in planta) in tissue of a plant leaf from a plant in a plant population, wherein the Raman spectra includes one or more peaks characteristic of carotenoids and/or proteins;
(b) obtaining a Raman spectra of carotenoids and/or proteins in vivo and in situ (i.e., in planta) in tissue of a plant leaf from a control plant, wherein the Raman spectra includes one or more peaks characteristic of carotenoids;
(c) comparing intensity of the one or more peaks characteristic of carotenoids and/or proteins from the Raman spectra obtained from the plant in the plant population with intensity of the one or more peaks characteristic of carotenoids from the Raman spectra obtained from the control plant; and;
(d) determining if there is an increase in the relative intensity of one or more peaks characteristic of carotenoids and/or proteins from the Raman spectra obtained from the plant in the plant population,
wherein an increase in relative intensity of the one or more peaks characteristic of carotenoids and/or proteins from the Raman spectra obtained from the plant in the plant population is indicative of an early stage infection.

In another aspect, the present invention provides a method of determining an elicitor response factor (ERF). The ERF provides a quantitative measure of the degree of pattern-triggered immunity (PTI) as determined by the difference Raman spectrum. The use of ERF as a diagnostic Raman signature for early PTI will provide an important tool for the identification of plants with early pathogen infection and facilitate effective disease management. In accordance with this aspect, the method comprises the following steps.

Step 1: After pre-processing (cosmic ray removal, Savitsky-Golay smoothing, and polynomial background subtraction), the mean of 60 Raman spectra from each individual biological sample was obtained in the Raman shift spectral range of 400 cm⁻¹-1,700 cm⁻¹.

Step 2: The difference of the mean spectra, as obtained in Step 1 above, between elicitor-treated and mock control-treated samples were subsequently derived to highlight different Raman spectral regions with positive values.

Step 3: A p-value plot was obtained using a t-test to evaluate the statistical significance of differential Raman spectra obtained in Step 2. Differential Raman spectral regions where p-value <0.05 were noted. The p-value plot was corrected by including an estimation of the positive false discovery rate (pFDR) and applying the multiple-hypothesis testing principle.

Step 4: The area under the curve of the differential Raman spectral region which has a positive value and also represents the corrected p-value <0.05 of Step 3 is defined as the ERF, which we used to measure the level of PTI response. A higher ERF value indicated a higher level of the elicitor-induced immune response.

Step 5: All the positive spectral regions contributing towards ERF were tabulated.

In some embodiments, the tissue of the plant leaf is a leaf blade. In other embodiments, the tissue of the plant leaf blade is proximal to the site of infection. In some embodiments, the one or more peaks characteristic of carotenoids and/or proteins in the Raman spectra are selected from the group of peaks consisting of 1001 cm⁻¹, 1151 cm⁻¹ and 1521 cm⁻¹ for carotenoids and 1550 cm⁻¹ for proteins. In other embodiments, the Raman spectra are obtained using near-infrared excitation wavelength. In some embodiments, the near-infrared excitation wavelength is 830 nm. In other embodiments, obtaining the Raman spectra is non-invasive and non-destructive to the tissue of the plant leaf. In other embodiments, the Raman spectra are obtained from a plant leaf during regular sampling, e.g., daily sampling. In some embodiments, the Raman spectra are obtained from a plant leaf at about 10 hours to about 24 hours after a suspected pathogen attack.

In another aspect, the present invention provides a method of containing the spread of pathogen infection of plants in a plant population comprising: (a) diagnosing early stage infection of a plant in a plant population according to a method described herein and (b) removing the early stage infected plants from the plant population.

### BRIEF DESCRIPTION OF THE FIGURES

FIGS. 1a-1e show experimental procedures for Raman spectroscopic analysis of elicitor-treated plants. (FIG. 1a) A 5-week-old Arabidopsis thaliana plant with leaves numbered according to the developmental stage. Leaf 1 is the first true leaf that emerged after the cotyledons. Scale bar =2 cm. (FIG. 1b) The abaxial (bottom) side of the leaf to be syringe-infiltrated. Leaf was inoculated by infiltration with a blunt syringe. (FIG. 1c) Images of plants after 24 hpi. Scale bar=2 cm. (FIG. 1d) Images of callose deposits in aniline-blue stained leaves that were previously infiltrated with mock (H₂O), 1 μM flg22 or elf18 for 24 h. Scale bar=20 μm. (FIG. 1e) Schematic of a custom-built NIR Raman spectroscopy used to detect secondary metabolites in plants. Insert shows the infiltrated sites and the leaf discs taken from the proximal region of the sites for analysis

FIGS. 2a-2d show effects of sample location and elicitor treatment time on changes in Raman spectra. (FIG. 2a) The abaxial (bottom) side of a leaf to be infiltrated with elicitor. Elicitor (1 μM) was infiltrated with a needle-less syringe at two locations (indicated as two small circles) nearest to the petiole. Leaf samples were taken from the proximal (PR) and distal (DR) regions at 24, 48, and 72 hpi and analyzed by Raman spectroscopy. (FIG. 2b, FIG. 2c) A group of 3 panels was shown for either the DR or the PR sample. Upper panel: mean spectra of control (black) and elicitor-treated samples (flg22; elf18). Middle panel: Difference spectrum between the mean spectrum of the elicitor-treated sample versus that of the mock-treated sample. A value above zero indicates that the difference is a positive value whereas a value below zero indicates a negative value. The number inside the panel indicates the average ERF (Elicitor Response Factor, ×10⁴) value obtained from 4 (b) or 3 (c) independent experiments. Lower panel: t-test was used to evaluate statistically significant differences between elicitor-infiltrated and H₂O-infiltrated (mock) samples and the results expressed as p-value. A value below the line indicates p-value <0.05. (FIG. 2d) Changes in ERF after the time of infiltration. Left, flg22 (spots), n=4 independent biological samples; Right, elf18 (spots), n=3 independent biological samples. PR, proximal region; DR, distal region. Results represent the average values of 3-4 independent biological experiments with standard deviation (s.d.). Bars with different letters above are significantly different according to the Fisher least significant difference test (p<0.05).

FIGS. 3a-3c shows induction of immune response genes by elicitors. Leaves were collected at the indicated time points after infiltration with 1 μM elicitor (flg22, middle bar; elf18, right bar) and transcript levels were determined by RT-qPCR. Transcript levels of Act2 (Actin2, At3g18780) were used for normalization. Expression values of elicitor treated samples were shown relative to the levels obtained with mock treatment (left bar) and the average values of 3 technical replicates were shown with standard deviation (s.d.). Similar results were obtained from three independent biological experiments. (FIG. 3a) Induction of expression of immune-response related genes after flg22 and elf18 treatment. Abbreviation: WRKY53 (At4g23810), early senescence WRKY networking regulator; WRKY29 (At4g23550); WRKY30 (At5g24110); ZAT12 (At5g59820), ZINC FINGER OF ARABIDOPSIS THALIANA12; PR1 (At2g14610), PATHOGENESIS-RELATED GENE1); PDF1.2 (At5g44420), PLANT DEFENSIN; MPK3 (At3g45640), Mitogen-activated Protein Kinase3; SANI) (At2g28390), MONENSIN SENSITIVITY1 (MON1), SAND family protein. SAND) was used as a control as its expression was not affected by elicitor infiltration. hpi, hours post-infiltration. (FIG. 3b) Effect of flg22 and elf18 treatment on the expression of tryptophan-metabolism related genes. MYB122 (At1g74080), MYB51 (Atlg18570), MYB34 (At5g60890), (YP79B2 (At4g39950), CYP79B3 (At2g22330), CYP83B1 (At4g31500), (YP71A13 (At2g30770), CYP71B15/pad3 (At3g26830). (FIG. 3c) A diagram showing tryptophan metabolism pathways and related structural and regulatory genes. Treatment of flg22 induces MYB122, MYB51, and MYB34 (Frerigmann et al., 2016).

FIG. 4a-4b show Raman spectral analysis and calculation of the elicitor response factor (ERF). (FIG. 4a) Top panel: Raw Raman spectra obtained from mock control and elicitor-treated samples after spectral pre-processing (cosmic ray removal, Savitsky-Golay smoothing, and polynomial background subtraction). Second panel: Mean Raman spectra obtained from control and elicitor-treated samples (Mean plots). Third panel: Difference Raman spectrum of elicitor-treated versus control samples (Difference of Mean plot). Bottom panel: False discovery rate adjusted p-value calculation by performing t-test on Raw Raman spectra. ERF was defined as the area below the Difference Raman spectrum curve, shown above in the third panel, after selecting positive spectral regions corresponding to adjusted p-value <0.05 (FIG. 4b) MATLAB programming language script used to calculate ERF.

FIGS. 5a-5b show Raman spectroscopic analysis of WT and mutant plants. (FIG. 5a) Raman spectra of WT Col-0 and mutant plants (fls2, efr-2, bak1, bik1, and rbohd) at 24 hr after flg22 and elf18 treatment and mock control. Top panel: Images of plants 24 hpi. For each elicitor experiment (flg22, and elf18) a group of 3 panels was shown. Scale bar=2 cm. Upper panel: a mean plot of all 60 spectra, taken from control (black) or elicitor-treated samples (grey); Middle panel: the difference spectrum between the mean spectrum of the elicitor-infiltrated sample and that of the H₂O-infiltrated (mock) sample. A value above the line indicates that the difference is a positive value whereas a value below the line indicates a negative value. The number inside the panel indicates the average ERF (Elicitor Response Factor, ×10+) value obtained from 4 independent experiments. Lower panel: t-test was used to evaluate statistically significant differences between elicitor-infiltrated and H₂O-infiltrated (mock) samples and the results expressed as p-value. A value below the line indicates p-value <0.05. Plants: Col-0, WT Arabidopsis thaliana; fls2, FLAGELLIN-SENSING2 (At5g46330); efr-2, EF-Tu receptor (At5g20480); bak1, BRASSINOSTEROID INSENSITIVE-ASSOCIATED KINASE1 (At4g33430); bik1, BOTRYTIS-INDUCED KINASE1 (At2g39660); and rbohd, NADPH respiratory burst oxidase protein D (At5g47910). (FIG. 5b) ERF of WT and mutants treated with either flg22 or elf18. Results show average ERF values along with s.d. Bars with different letters above are significantly different according to the Fisher least significant difference test (p<0.05). Left: flg22 (spots); Right, elf18 (spots). n=4 independent replicates.

FIG. 6a-6b shows determination of callose deposition in WT and mutant plants after elicitor treatment. Callose deposition in the rosette leaves of 5-week-old Arabidopsis WT (Col-0), PAMP-associated mutants (fls2, efr-2, bak1, bik1, rbohd), and wounding-related mutant (glr3.3glr3.6) treated for 24 hr with H₂O, or 1 μM flg22, or 1 μM elf18. (FIG. 6a) Representative pictures of aniline-blue stained callose fluorescence; Scale bars=20 μm. (FIG. 6b) Quantification of callose deposition by aniline blue-staining at 24 hr after infiltration of mock (black bar), 1 μM flg22 (middle bar), and 1 μM elf18 (right bar). Values shown are mean±s.d. of 25-30 surveyed leaf regions. Each region covered an area of 5,996 μm². For each experiment, the number of callose was measured in 2 infiltrated plants with about 4-5 leaf regions surveyed per plant. This gave a total of about 8-10 data points per experiment. The experiment was repeated with 3 independent biological samples. The graph shows average value with n=25-30.

FIGS. 7a-7b shows induction of PTI marker genes in response to 1 μM elicitor (flg22 and elf18) in mutants. Arabidopsis thaliana ecotype Columbia (Col-0), fls2 (salk_062054), efr-2 (salk_068675), bak1 (salk_116202), bik1 (salk_005291), rbohd (salk_083046), and glr3.3/3.6 loss-of-function mutants (FIG. 7a) and pub12/13 and pub25/26 loss-of-function mutants (FIG. 7b). 5-week-old plants were treated with flg22 or elf18, at a final concentration of 1 μM, and leaf samples collected at 0.5 or 6 hpi to analyze the expression of PR1 (At2g14610), pathogenesis-related gene 1; WRKY53 (At4g23810), WRKY29 (At4g23550), WRKY30 (At5g24110), ZAT12 (At5g59820); a zinc finger protein, PMR4 (At4g03550), PAD3 (At3g26830), MPK3 (At3g45640), and MYB51 (At1g18570) as PTI marker genes by qRT-PCR. The relative fold change±s.d. of the PAMP-responsive genes was obtained from a comparison between the PAMP-treated plants and mock-treated plants with the reference gene act1 (At3g18780). The average values of 3 technical replicates were shown with s.d., and the similar results were obtained from three independent biological experiments.

FIGS. 8a-8d show Raman spectroscopic analysis of WT and pub12pub13 and pub25pub26 double mutants. (FIG. 8a) Top panel: Images of plants 10 hpi. For each elicitor experiment (flg22, and elf18) a group of 3 panels was shown. Raman spectra were acquired at 10 hpi with 1.0 μM flg22 and elf18 treatment and mock control (black peak). Upper panel: a mean plot of all 60 spectra, taken from control (black) or the elicitor-treated samples (grey, flg22; grey, elf18); Middle panel: the difference spectrum between the mean spectrum of the elicitor-infiltrated sample and that of the H₂O-infiltrated (mock) sample. A value above the line indicates that the difference is a positive value whereas a value under the line indicates a negative value. The numbers inside the panel indicate average ERF values. Lower panel: t-test was used to evaluate statistically significant differences between elicitor-infiltrated and H₂O-infiltrated (mock) samples and the results expressed as p-value. A value below the line indicates p-value <0.05. Plants: Col-0, WT Arabidopsis thaliana; pub 12pub13, U-box E3 ubiquitin ligase PUB12/13 (At2g28830 and At3g46510, respectively); fls2, FLAGELLIN-SENSING2 (At5g46330); pub25pub26, U-box E3 ubiquitin ligase PUB25/26 (At3g19380 and Atlg49780, respectively); bik1, BOTRYTIS-INDUCED KINASE1 (At2g39660). Scale bars=2 cm. (FIG. 8b) ERF (Elicitor Response Factor) of WT and mutants treated with either flg22 or elf18. Results show mean ERF values along with s.d. Bars with different letters above are significantly different according to the Fisher least significant difference test (P<0.05). Left: flg22 (spots); Right, elf18 (spots). n=4 independent biological replicates. (FIG. 8c) Representative images of callose deposits (bright spots) in treated samples; Scale bars=20 μm. (FIG. 8d) Quantification of callose deposits in WT (Col-0) pub12/13, fls2, pub25/26, and bik1 mutant leaves. The number of callose fluorescence spots was used to determine the number of callose deposits. Left: mock (black spots); Middle: flg22 (spots); Right, elf18 (spots). Values shown are mean±s.d. of 25-30 surveyed leaf regions. For each experiment, the number of callose deposits was measured in 2 infiltrated plants with about 4-5 leaf regions surveyed per plant. The value for each leaf region gives the number of callose deposits per 5,996 μm². This gave a total of about 8-10 data points per experiment. The experiment was repeated with 3 independent biological samples. The graph shows average value with n=25-30. Bars with different letters above are significantly different according to the Fisher least significant difference test (p<0.05).

FIG. 9 shows difference spectra with annotated peaks at 24 hpi for Arabidopsis treated with flg22 (left panel) or elf18 (right panel).

FIGS. 10a-10b show changes in carotenoid levels in Arabidopsis leaf samples treated with elicitor for 24 hr. (FIG. 10a) Raman spectra of carotenoids from leaves with mock (left bar) and elicitor (flg22 middle bar, and elf18 right bar) treatment for 24 hr. Raman shift at 1,003 cm-1, 1,151 cm⁻¹ and 1,522 cm⁻¹ are attributed to carotenoids. n=3 independent biological samples. (FIG. 10b) Changes of total carotenoids levels in Arabidopsis leaves after treatment with mock control (left bar), flg22 (middle bar), and elf18 (right bar) for 24 hr (24 hpi). Carotenoid levels were determined as described in Materials and Methods. Values shown are mean±s.d. of extracted total carotenoids. For each experiment, the quantitation of total carotenoids was measured in 3-5 infiltrated plants per experiment. The experiment was repeated with 4 independent biological samples, and this gave a total of about 15-20 data points. **, *** significantly different from the mock control at p<0.001, and p<0.0001 (t-test), respectively.

FIGS. 11a-11b shows the general carotenoid metabolic pathway in Arabidopsis and expression changes of carotenoid biosynthetic genes after elicitor treatment. (FIG. 11a) A schematic diagram showing carotenoids biosynthesis and cleavage pathways in plants. (FIG. 11b) Expression levels of carotenoids biosynthetic genes at various times after elicitor treatment. Enzymes and corresponding Arabidopsis mutant names (in italics) are listed. PSY, phytoene synthase (At5g17230); PDS, phytoene desaturase (At4g14210); Z-ISO, z-carotene isomerase (At1g10830); ZDS, z-carotene desaturase (At3g04870); CRTISO, carotenoid isomerase (At1g06820); εLCY, Lycopene ε-cyclase (LUT2, At5g57030); βLCY, Lycopene β-cyclase (At3g10230); CYP97A3, (LUT5, At1g31800); CYP97C1, (LUT1, At3g53130); CYP97B3, (At4g15110); BCH1, β-carotene hydroxylase 1 (At4g25700); BCH2, β-carotene hydroxylase 2 (At5g52570); CCS, COPPER CHAPERONE FOR SOD1 (At1g12520); ZEP, Zeaxanthin epoxidase (ABA1, At5g67030); NPQ, NON-PHOTOCHEMICAL QUENCHING1 (At1g08550), NXS, neoxanthin synthase (ABA4, At1g67080); NCED3, 9-cis-epoxycarotenoid dioxygenase (At3g14440); ABA2, ABA DEFICIENT 2 (At1g52340); AAO3, ABSCISIC ALDEHYDE OXIDASE3 (At2g27150); CCD1, carotenoid cleavage dioxygenase1 (At3g63520); CCD4, At4g19170; (CD7, At2g44990; (CD8, At4g32810; D) 27, β-carotene isomerase (At1g64680); (YP711A, (At2g26170). Transcript levels of Act2 (Actin2, At3g18780) were used for normalization. Expression values of elicitor treated samples (flg22 middle bar; elf18 right bar) were shown relative to the levels obtained with mock treatment (grey bar) and the average values of 3 technical replicates were shown with s.d. The similar results were obtained from two independent biological experiments.

FIG. 12 shows the spread in Raman peak intensities analysed by histogram plots: Besides the main carotenoid Raman peak at wavenumber 1521 cm⁻¹ (right panel), it was observed that Raman shift wavenumber at 1550 cm⁻¹ (left panel) could also clearly distinguish between mock and elicitor treated samples. Histogram analysis was performed to make sure that data was not skewed by an outlier. All Raman shift wavenumbers mentioned in the Table 1 were analysed using his histogram analysis.

FIGS. 13a-13d show Raman spectra analysis of plants treated with pathogens. (FIGS. 13a, 13b) Pathogens (Pseudomonas syringae pv. Tomato D (3000, a; Xanthomonas campestris pv. campestris, b) were infiltrated with a needle-less syringe into Arabidopsis and Choysum respectively. Leaf samples taken from the proximal regions at 24, 48, and 72 hpi were analyzed by Raman spectroscopy. Upper panel: mean spectra of control (black) and pathogen-treated samples (grey). Middle panel: Difference spectrum between the mean spectrum of the pathogen-treated sample versus that of the mock-treated sample. A value over the line indicates that the difference is a positive value whereas a value under the line indicates a negative value. The number inside the panel indicates the average IRF (Infection Response Factor, ×10⁴) value obtained from 3 independent experiments. Lower panel: t-test was used to evaluate statistically significant differences between pathogen-infiltrated and mock-infiltrated samples and the results expressed as p-value. A value under the line indicates p-value <0.05. (FIGS. 13c, 13d) Changes in IRF after the time of infiltration. Left, Pseudomonas syringae pv. Tomato DC3000 (grey spots), n=3 independent biological samples; Right, Xanthomonas campestris pv. campestris (grey spots), n=3 independent biological samples. Results represent the average values of 3 independent biological experiments with standard deviation (s.d.). Bars with different letters above are significantly different according to the Fisher least significant difference test (p<0.05).

FIGS. 14a-14b shows changes in secondary metabolites (carotenoid and callose) levels in Brassica rapa (Choysum) leaf samples treated with Xanthomonas campestris pv. campestris for 24 hr and 48 hr. (FIG. 14b) Raman spectra of carotenoids from leaves with mock and elicitor treatment for 24 hr. Raman shift at 1,001 cm⁻¹, 1,151 cm⁻¹ and 1,521 cm⁻¹ are attributed to carotenoids. n=3 independent biological samples. (FIG. 14a) Representative images of aniline-blue-stained callose deposition in the mock and Xcc infiltrated Choysum leaves at different time points; Scale bars=20 μm.

FIG. 15 shows Raman peak assignments of bacteria infected plants. The left panel difference spectra with annotated peaks at 24 hpi for Arabidopsis infected with Pseudomonas syringae. The left panel difference spectra with annotated peaks at 24 hpi for Choysum infected with Xcc.

### DETAILED DESCRIPTION OF THE INVENTION

The present invention relates to the use of Raman spectroscopy for the real time detection and quantitation of innate immunity response in plants. More specifically, the present invention provides Raman spectroscopy as a tool for rapid, non-invasive, and early detection and quantitation of plant innate immune response. Rapid detection of pathogen-infected plants is important and first in plant disease management. As used herein, “real time detection” refers to methods or systems which can provide information about plant disease as it happens.

In one embodiment, the methods described herein are particularly suited for the real time detection and quantitation of innate immunity response in plants. in urban farming.

In another embodiment, the methods described herein, with slight modifications, can be used for the real time detection and quantitation of innate immunity response in plants in open farming, i.e., commercial farming in open fields. Such slight modifications would be known to the skilled artisan on the basis of the description herein and may include slight optical modification we the use of optical filters. The Raman system described herein can be further miniaturized and attached to, e.g., a robotic arm or other mobile machinery. The Raman system described herein or further miniaturized can also be integrated in drones.

Plant transmembrane receptors bind to biomolecules carrying pathogen-associated molecular patterns (PAMP) and initiate signaling of the innate immune pathway, referred to as PAMP-triggered immunity (PTI) response. For example, when Arabidopsis plants were challenged with flg22 and elf18 elicitors, it was observed that the mock (control) and elicitor treated plants could be differentiated using their Raman spectral fingerprints. Using the difference Raman spectrum from mock and elicitor treated Arabidopsis plants, and factoring in the p-value to calculate the statistical significance of observed Raman peaks, the Elicitor Response Factor (ERF) is derived as a quantitative measure of the early PTI response. ERF values at 24 hour post infection (hpi) were found to be significantly higher than ERF values at 48 hpi.

Among the various Raman spectral bands contributing towards the ERF value, the most significant changes were observed in those associated with the carotenoids and proteins. To validate these results, we investigated several characterized Arabidopsis PTI mutants. Compared to wild type (WT), positive regulatory mutants had ERF values close to zero whereas negative regulatory mutants at early time points had higher ERF values.

As observed with elicitor treatments, an analogous Infection Response Factor (IRF) was derived as a quantitative measure to detect early PTI response in Arabidopsis and Choysum plants infected with bacterial pathogens. High IRF values at 24 hpi were observed when compared to 48 hpi. The Raman spectral bands contributing towards the high IRF value in plants, as shown with Arabidopsis and Choysum, were largely identical to the ERF Raman spectral bands.

As shown herein, Raman spectroscopy is a convenient tool for rapid screening of biotic stress in plants, such as Arabidopsis and leafy vegetables. It can be used to screen Arabidopsis PTI-mutants to advance plant biology and for non-invasive early diagnosis of pathogen-infected crop plants.

The present invention provides Raman spectroscopy as a tool for rapid, non-invasive, and early diagnosis of pathogen infected crop plants. Elicitor-triggered early pattern triggered immunity shows peak characteristics of carotenoids and/or proteins. Thus, a transient increase (5-10%) in carotenoids and/or proteins can be taken as markers for identifying an early onset of infection in a plant population. The transient increase in carotenoids has also been confirmed by the direct chemical analysis, i.e, spectrophotometer. Any such early stage infected plants may then be physically removed for containing the spread of infection. The transient increase in carotenoids is evident at 1001 cm⁻¹, 1151 cm 1, and 1521 cm⁻¹, and the transient increase in proteins is evident at 1550 cm⁻¹. These transient increases in carotenoids and/or proteins are an early event in pattern-triggered immunity. The carotenoid and/or protein increase is detected at about 10-24 hrs upon receptor activation and way before the manifestation of any disease symptoms.

The present invention relates to a rapid method to monitor early Pattern-Triggered Immunity (PTI) response in plants as shown herein for Arabidopsis and Choysum. Using flg22 and elf18 to elicit PTI, the difference Raman spectrum between mock- and elicitor-treated Arabidopsis plants was measured at, e.g., 10-24 hours post infection. A significant increase in intensity of vibrational bands associated with carotenoids and/or proteins was observed as an early response in treated samples. Other Raman peaks that change upon plant infection are shown in Tables 2-5 herein. This effect was confirmed by direct chemical analysis. From the difference Raman spectrum and the p-value at each Raman shift, the Elicitor Response Factor (ERF) was derived as a quantitative measure of the response. Raman spectroscopy was also used to investigate several Arabidopsis PTI mutants. Compared to WT, positive regulatory mutants have ERF values close to zero whereas negative regulatory mutants show carotenoid and/or protein increases at early time points and have higher ERF values.

The invention uses Raman spectral signature of plant metabolites, specifically carotenoids and/or proteins, as biomarkers for an early, real-time diagnosis of pathogen infection in growing plants in a non-invasive or non-destructive way, wherein plants need not be “destroyed” in order to detect pathogen infection. Transient increases (about 5% to about 10%) in carotenoids and/or proteins are detected before manifestation of any disease symptoms, highlighting carotenoids and/or proteins as indicator metabolites for identifying early onset of infection, e.g., at about 10 hours to about 24 hours after receptor activation, and Raman spectroscopy as a predictive tool for early diagnosis. Raman-based signatures can be used in a hand-held Raman spectroscope to detect the early onset of infection in plants. Any Raman spectroscope can be used in the invention for detecting early infection in plants.

The early, real-time diagnosis of early infection in plants, e.g., in individual plants in a plant population, enables the physical removal of early stage infected plants and thus contain the spread of infection. Containing the spread of infection will ensure yield of plants, such as leafy vegetables growing in, particularly, artificial urban farming settings.

Thus, in one aspect, the present invention provides a method of diagnosing early onset of pathogen infection of a plant. In accordance with this aspect, the method comprises:
(a) obtaining a Raman spectra of carotenoids and/or proteins in vivo and in situ (i.e., in planta) in tissue of a plant leaf from a plant in a plant population, wherein the Raman spectra includes one or more peaks characteristic of carotenoids and/or proteins;
(b) obtaining a Raman spectra of carotenoids and/or proteins in vivo and in situ (i.e., in planta) in tissue of a plant leaf from a control plant, wherein the Raman spectra includes one or more peaks characteristic of carotenoids and/or proteins;
(c) comparing intensity of the one or more peaks characteristic of carotenoids and/or proteins from the Raman spectra obtained from the plant in the plant population with intensity of the one or more peaks characteristic of carotenoids and/or proteins from the Raman spectra obtained from the control plant; and;
(d) determining if there is an increase in the relative intensity of one or more peaks characteristic of carotenoids and/or proteins from the Raman spectra obtained from the plant in the plant population,
wherein an increase in relative intensity of the one or more peaks characteristic of carotenoids and/or proteins from the Raman spectra obtained from the plant in the plant population is indicative of an early stage infection.

In another aspect, the present invention provides a method of determining an elicitor response factor (ERF). The ERF provides a quantitative measure of the degree of pattern-triggered immunity (PTI) as determined by the difference Raman spectrum. The use of ERF as a diagnostic Raman signature for early PTI will provide an important tool for the identification of plants with early pathogen infection and facilitate effective disease management. As used herein, the terms “high ERF” or “higher ERF” refers to an ERF having a value of at least 1×10⁴. In accordance with this aspect, the method comprises the following steps.

Step 1: After pre-processing (cosmic ray removal, Savitsky-Golay smoothing, and polynomial background subtraction), the mean of 60 Raman spectra from each individual biological sample was obtained in the Raman shift spectral range of 400 cm⁻¹-1,700 cm⁻¹.

Step 2: The difference of the mean spectra, as obtained in Step 1 above, between elicitor-treated and mock control-treated samples were subsequently derived to highlight different Raman spectral regions with positive values.

Step 3: A p-value plot was obtained using a t-test to evaluate the statistical significance of differential Raman spectra obtained in Step 2. Differential Raman spectral regions where p-value <0.05 were noted. The p-value plot was corrected by including an estimation of the positive false discovery rate (pFDR) and applying the multiple-hypothesis testing principle.

Step 4: The area under the curve of the differential Raman spectral region which has a positive value and also represents the corrected p-value <0.05 of Step 3 is defined as the ERF, which we used to measure the level of PTI response. A higher ERF value indicated a higher level of the elicitor-induced immune response.

Step 5: All the positive spectral regions contributing towards ERF were tabulated.

In some embodiments, the tissue of the plant leaf is a leaf blade. In other embodiments, the tissue of the plant leaf blade is proximal to the site of infection. In some embodiments, the one or more peaks characteristic of carotenoids in the Raman spectra are selected from the group of peaks consisting of 1001 cm⁻¹, 1151 cm⁻¹ and 1521 cm⁻¹, and/or one or more peaks characteristic of proteins is a peak at 1550 cm⁻¹. In other embodiments, the Raman spectra are obtained using near-infrared excitation wavelength. In some embodiments, the near-infrared excitation wavelength is 830 nm. In other embodiments, obtaining the Raman spectra is non-invasive and non-destructive to the tissue of the plant leaf. In other embodiments, the Raman spectra are obtained from a plant leaf during regular sampling, e.g., daily sampling. In some embodiments, the Raman spectra are obtained from a plant leaf at about 10 hours to about 24 hours after a suspected pathogen attack.

In another aspect, the present invention provides a method of containing the spread of pathogen infection of plants in a plant population comprising: (a) diagnosing early stage infection of a plant in a plant population according to a method described herein and (b) removing the early stage infected plants from the plant population.

In laser Raman spectroscopy, monochromatic laser light is directed onto a particular material to be tested. A sensitive detection system then detects light returning, or scattered, from the material. The majority of the light returning from the material is scattered elastically at the same wavelength of the original projected laser light. A very small fraction of the light returning from the material is scattered inelastically at a wavelength different from that of the original projected laser light in a manner known as Raman scattering. Raman scattered light is then separated from Rayleigh scattered light with the use of filters, optical gratings, prisms, and other wavelength selection techniques. The energy difference between scattered Raman light and the incident laser light, conventionally represented in wave numbers (cm⁻¹), is related to the vibrational, rotational, or librational states, or combinations thereof, of various molecules in the material being evaluated. Each of the peaks in the resulting Raman spectrum corresponds to a particular Raman active vibration of a molecule or a component thereof. The Raman energy shift is independent of the wavelength of the directed laser light. That is, the energy difference corresponding to the elastically and inelastically scattered light for a particular material remains constant for that material. The characteristic results from Raman scattering can be used to locate, identify and quantify concentrations of a material. The absolute intensities of the resulting Raman peaks are directly related to the concentration of the Raman-active molecules in the material.

The present invention relates to the use of Raman spectroscopy to identify a biomarker that is associated with pathogen infection in plants, which then can be used for the early, real-time diagnosis of pathogen infection in plants and ultimately for containing infection of plants in a plant population. More specifically, the present invention relates to the use of a Raman spectral signature of plant metabolites, specifically carotenoids and/or proteins, as a biomarker for an early, real-time diagnosis of pathogen infection in plants in a non-invasive or non-destructive way in order to control pathogen infection in a plant population. The early, real-time diagnosis of pathogen infection in a plant in a plant population enables the removal of infected plants to limit the negative affect on the yield of growing plants, including leafy vegetables.

As shown herein, the concentration of carotenoids and/or proteins in leaf tissue is a biomarker for pathogen infection of plants. Carotenoids and/or proteins have been found to exhibit characteristic Raman scattering, the results of which show up in distinct spectral positions, signal strengths, and spectral widths. More specifically, and as shown herein using a Raman spectroscopy system, carotenoids exhibit strong characteristic Raman scattering signals at 1001 cm⁻¹, 1151 cm⁻¹ and 1521 cm⁻¹. Also as shown herein using a Raman spectroscopy system, proteins exhibit a strong characteristic Raman scattering signal at 1550 cm⁻¹. The intensity of the Raman signals are directly related to the concentration of carotenoids and/or proteins. Thus, an increase in the intensity of the Raman signals is indicative of an increase in the concentration of carotenoids and/or proteins, and a decrease in the intensity of the Raman signals is indicative of a decrease in the concentration of carotenoids and/or proteins. As shown herein, an increase in the concentration of carotenoids and/or proteins is indicative of pathogen infection in plants.

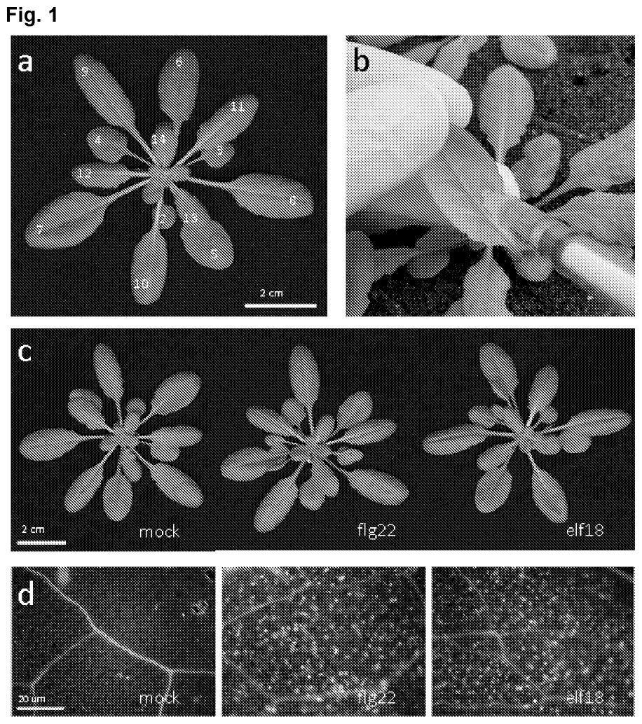

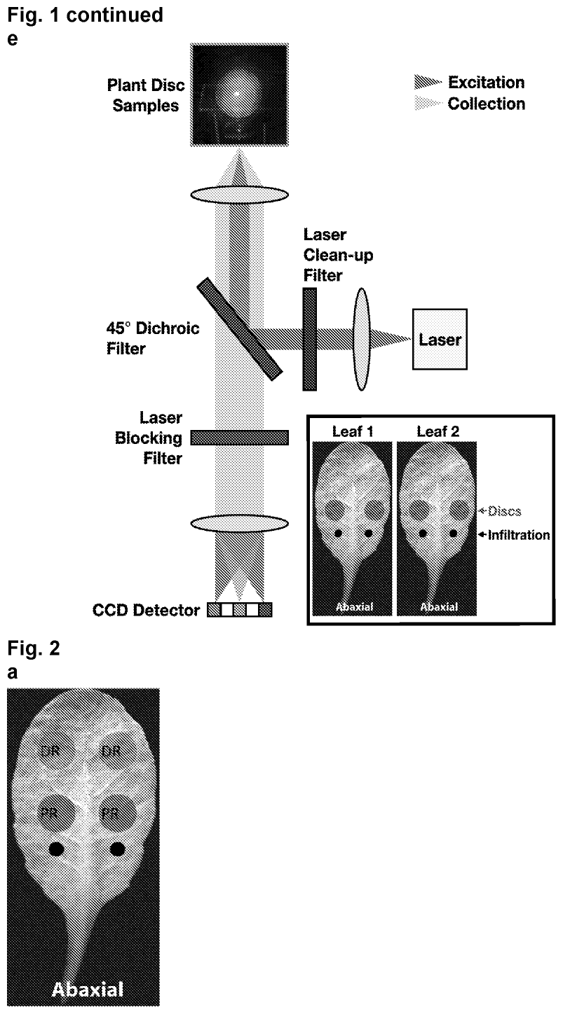

In some embodiments, Raman spectra are collected using a purpose-built Raman spectroscopy system shown in FIG. 1e. In one embodiment, a Raman spectroscopy system used herein is designed for 830 nm excitation. In general, the sample holder featured a 100 μm thick fused silica sampling window used for both excitation and collection of the Raman signal. An aspheric lens is used to focus the excitation light and collect the Raman scattered light. The lens is chosen with a depth of focus >1 mm so that Raman signal from the entire cross-section of a leaf is collected. The excitation laser used with this system is a fiber coupled laser (Innovative Photonic Solutions, USA) operating at 830 nm delivering approximately 100 mW of laser power to the sample. Light is delivered from the laser to collimating optics via a 105 micron core multimode fiber. The collimated light is passed through a Semrock MaxLine Laser Line 830 filter (Semrock Inc., USA) to remove any amplified spontaneous emission from the laser and any background generated within the delivery fiber. The filtered light is coupled into the optical path of the excitation lens by a Semrock long pass filter (Semrock Inc., USA) operated as a dichroic mirror. Collected light is passed back through the Semrock filter and then through an additional long pass filter to further attenuate Rayleigh scattered excitation light before being delivered to the spectrometer using an F #matching lens. Spectra are acquired using Kymera 328i spectrograph (Andor, UK) employing a 600 g/mm optical grating. While an embodiment of a Raman spectroscopy has been described, it is appreciated that any Raman spectroscopy system, including different excitation laser wavelengths or different excitation and collection optical design, or different Raman signal detector such as photodiodes or CCDs that can focus light on a plant leaf and collect Raman signal can be used for detecting biotic stress in plants. For Example in other embodiments, Raman spectra are collected using a purpose-built Raman spectroscopy system described in International patent application No. PCT/SG2021/050276 or Singapore patent application Ser. No. 10/202,004759U or described in International patent application No. PCT/SG2021/050275 or Singapore provisional patent application No. 10202004761W. In practice, Raman spectra are collected after infection in plant material such as a plant leaf. As an example, for each sample of plant leaf, 5 spectra are collected with an integration time of 10 s per spectrum from infected and mock-healthy leaves. Also, minimum of 3 sample spots on a leaf are selected for investigation. Cosmic ray events are identified in the 5 spectra, acquired with acquisition time of 10s each, and removed. After cosmic ray removal, the individual 10 s spectra are smoothed across wavelength using the Savitzky-Golay filter function (MATLAB Inc., USA) with 5th order polynomial and a frame length of 11. A representative sample spectrum is created by taking the mean value of the five filtered and smoothed spectra at each wavelength. The sample spectrum resulting from this processing contains Raman and fluorescence signal primarily from the leaf. To generate the leaf Raman spectra presented herein any residual fluorescence is removed by performing a positive residual style polynomial subtraction as described in reference (International patent application No. PCT/SG2021/050275). Calibration of the Raman shift is performed using a polystyrene sample with a well-known Raman spectrum (International patent application No. PCT/SG2021/050275). From the difference Raman spectra of mock and infected plants an Infection Response Factor (IRF) is derived as a quantitative measure to detect early infection in any crop as done in Arabidopsis and Choy Sum plants infected with bacterial pathogens whereby high IRF values indicate infection. The Raman spectral bands contributing towards the high IRF value can be analyzed and then annotated to confirm the role of various metabolites like carotenoids and proteins during infection during a particular infection in any crop.

In one embodiment, the concentration of carotenoids and/or proteins is determined within plant material. In some embodiments, the plant material is leaf material. In other embodiments, the leaf is the first true leaf, second true leaf, third true leaf, etc. In some embodiments, the leaf material is a leaf blade. In some embodiments, Raman spectra are collected from two leaf discs per leaf blade. In some embodiments, the one leaf disc is taken from the left side of the central leaf vein and the second leaf disc is taken from the right side of the central leaf vein. In other embodiments, a Raman spectrum is collected at one location in the middle of the leaf petiole. As shown herein an increase in the concentration of carotenoids and/or proteins increase in concentration in plant leaf tissue between about 10 hours to about 24 hours after pathogen infection.

Raman spectroscopy is faster and easier to use than other techniques used to determine concentrations of carotenoids and/or proteins in plant tissues, is non-invasive and not harmful to the plant, allows real-time measurements, measures the concentration of carotenoids and/or proteins in vivo and in situ (i.e., in planta) and enables focusing on small parts of plants for the analysis. These benefits of Raman spectroscopy enables the detection of early stage pathogen infection of plants in a plant population by Raman spectrometry before the manifestation of any disease symptoms in infected plants. The early diagnosis of pathogen infection in plants enables containing the spread of infection to other plants in the plant population. As shown herein, Raman spectroscopy can be used for early diagnosis of pathogen infection in plants and containing the spread of plant infections in all plant populations, including leafy vegetables.

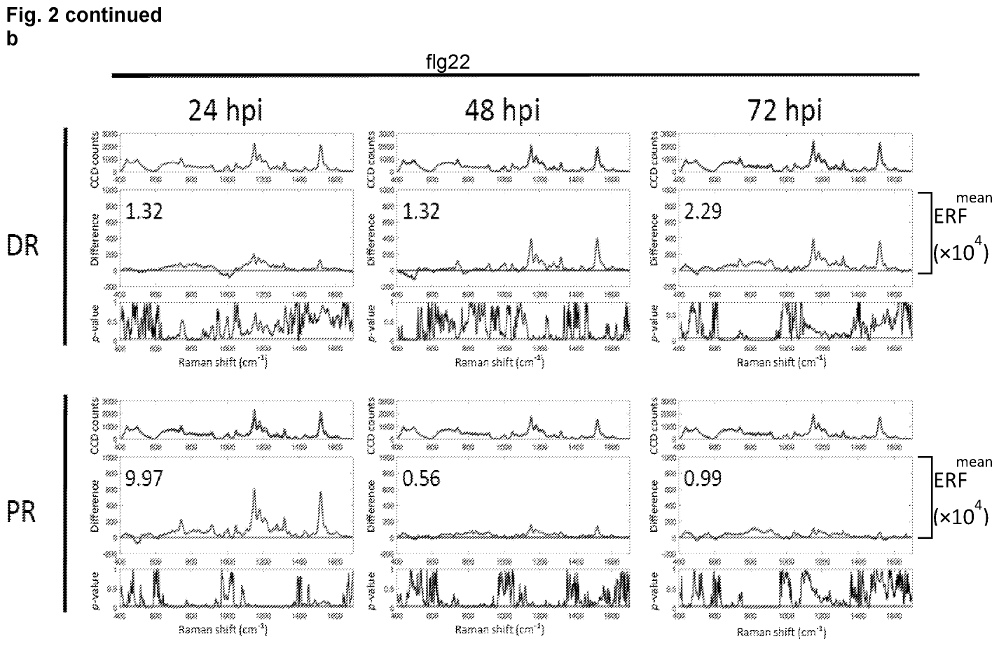

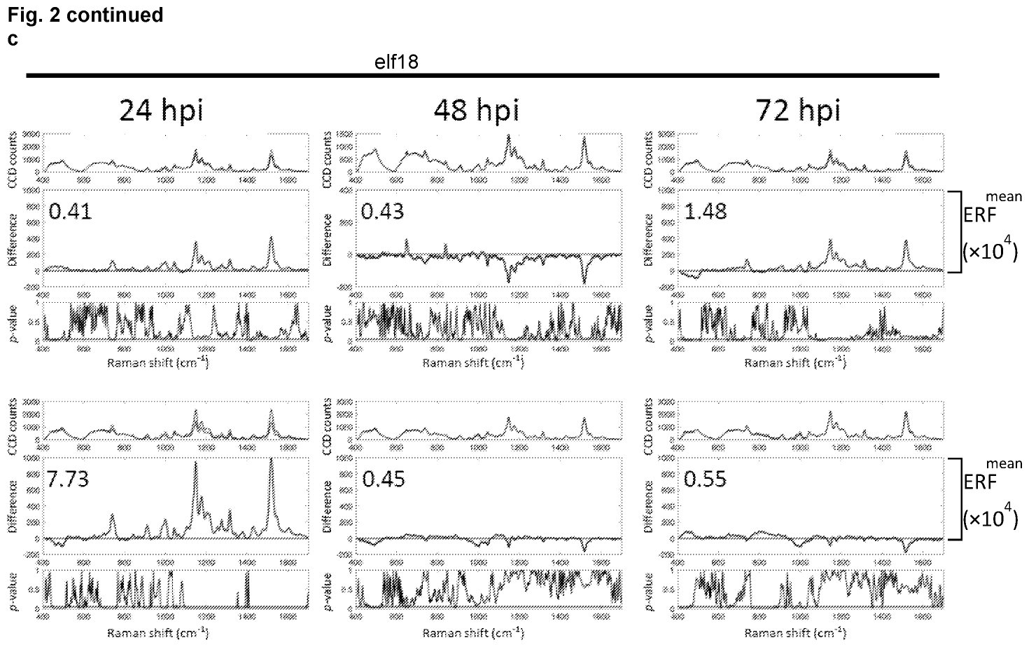

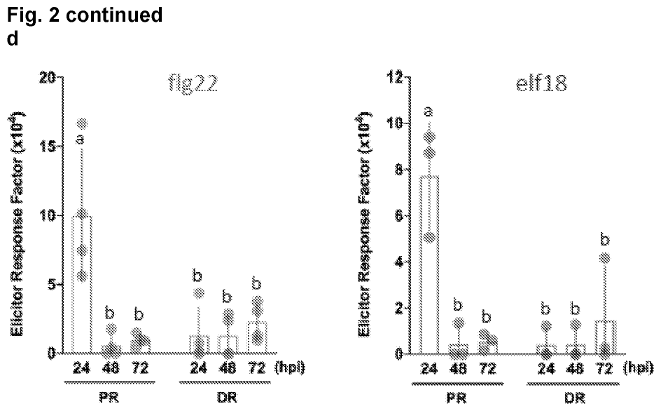

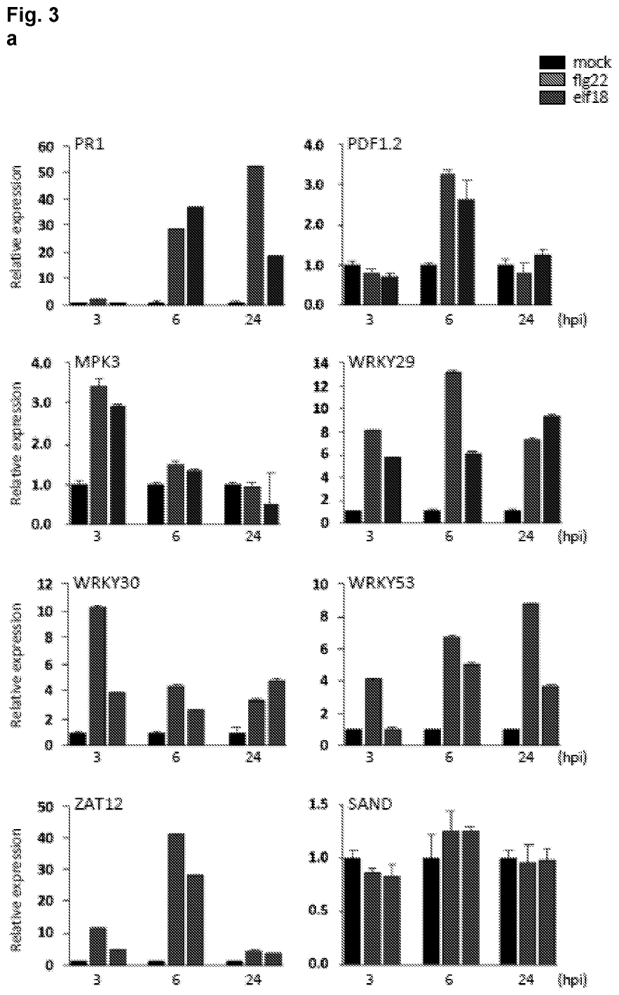

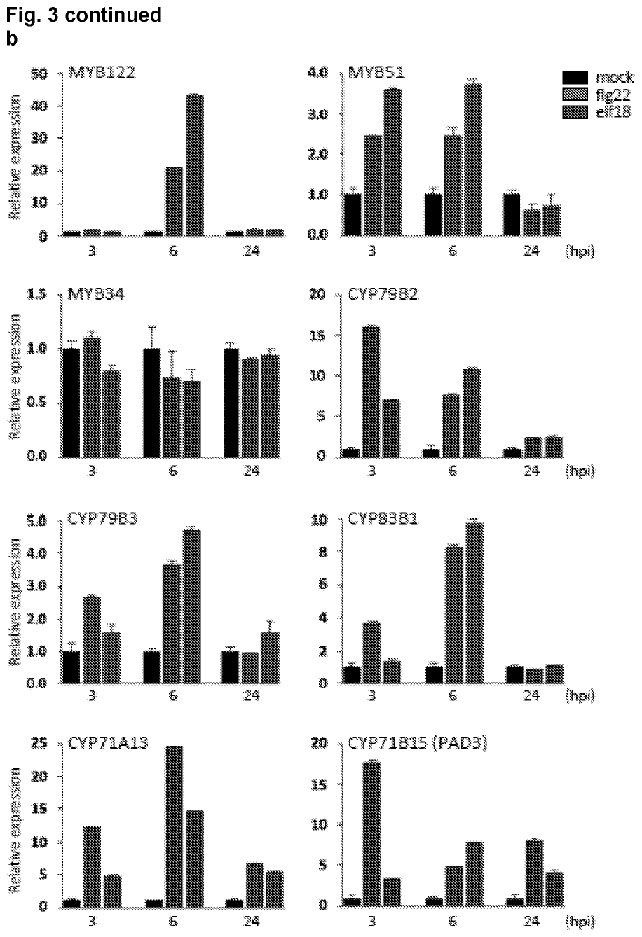

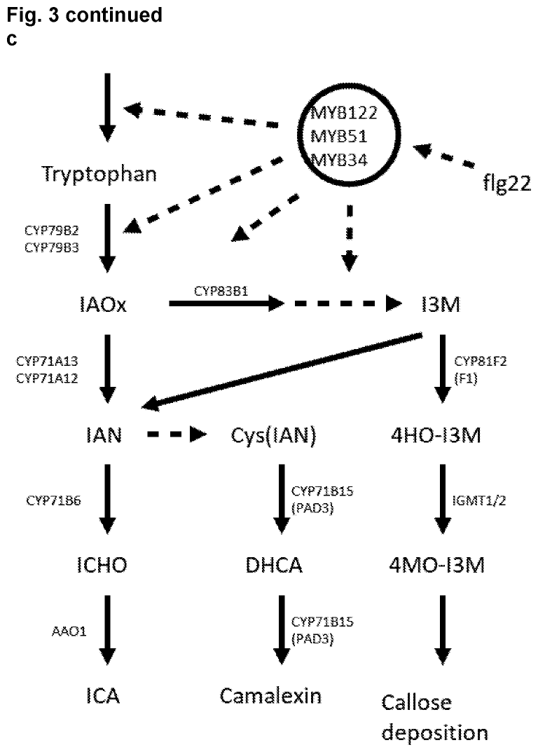

As shown in the Examples herein, infiltration of WT Arabidopsis leaves with elicitor can result in reproducible changes in the Raman spectra acquired from the leaf region proximal to the site of elicitor infiltration (FIGS. 1a-1e; FIGS. 2a-2d). Treatment with 1 μM elicitor induced expression of immune pathway genes at 3-6 hpi and callose deposition at 24 hpi (FIGS. 1a-1e; FIGS. 3a-3c). These results indicate that the elicitors were effective in producing a robust PTI in the treated plants and suggest that the Raman spectral changes are associated with this response.

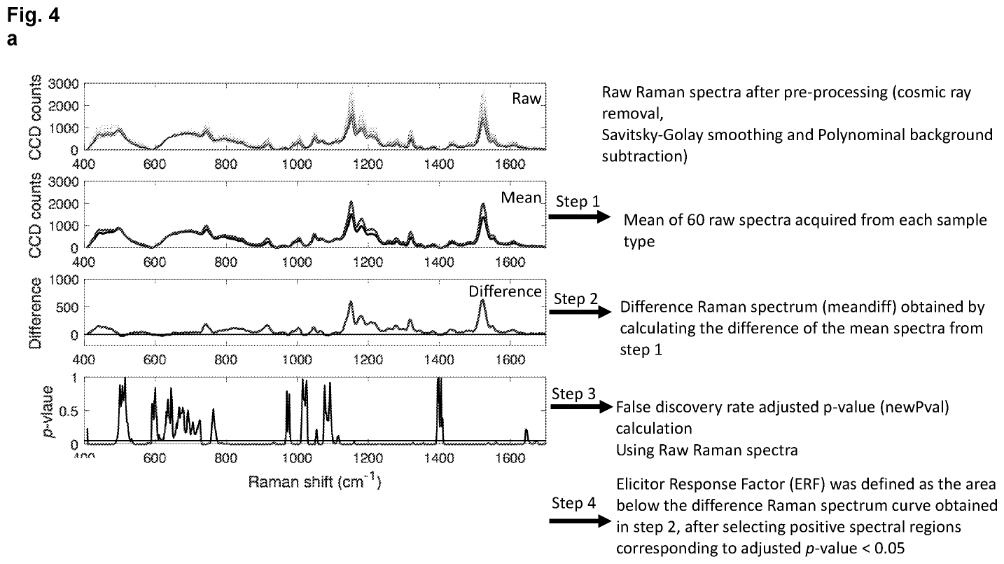

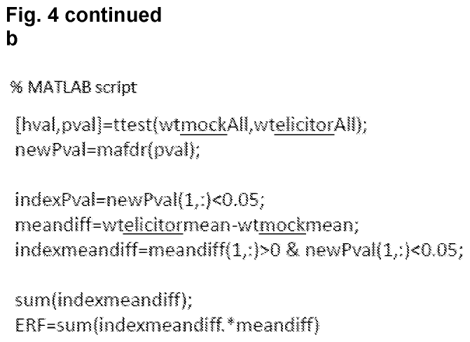

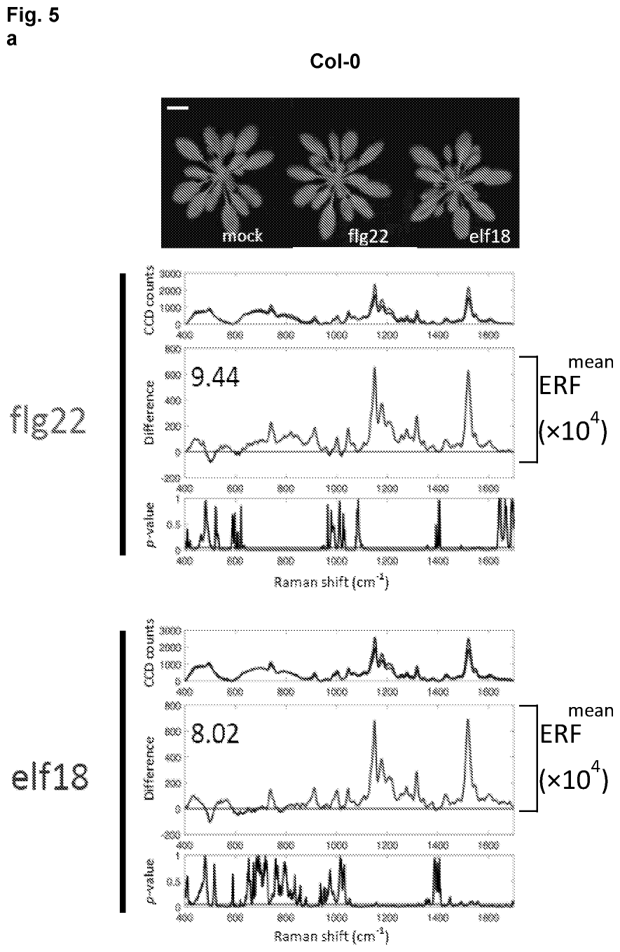

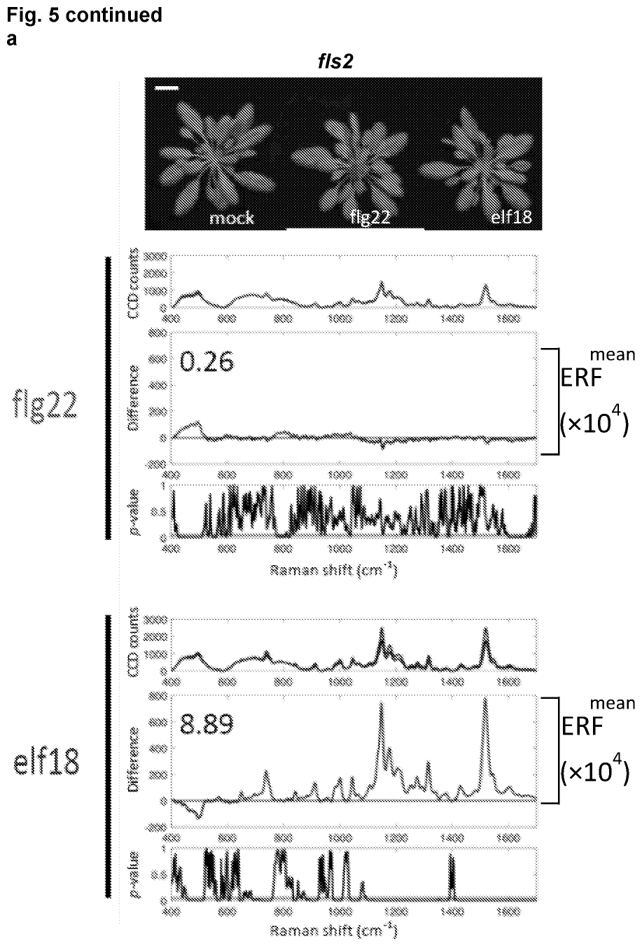

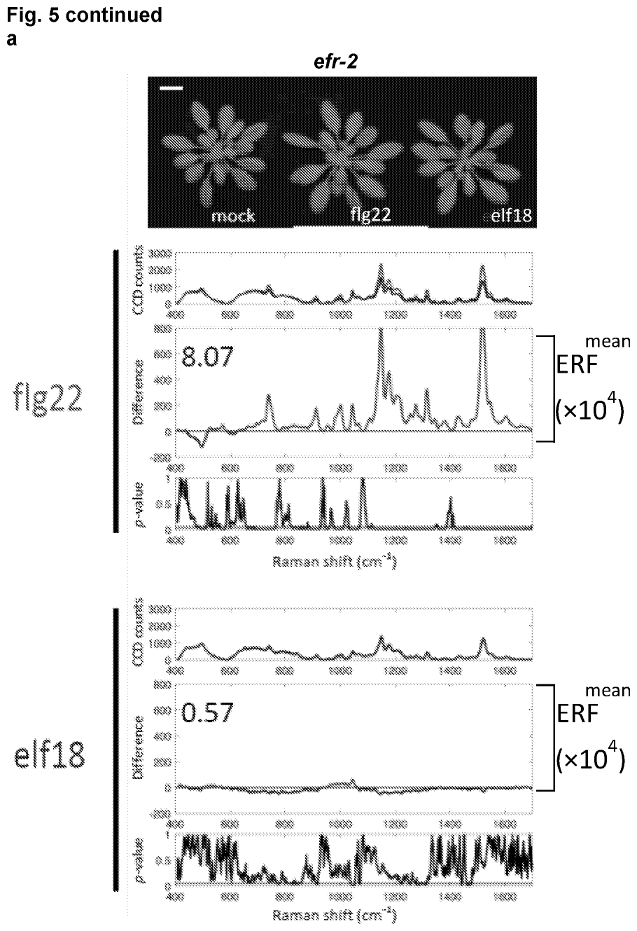

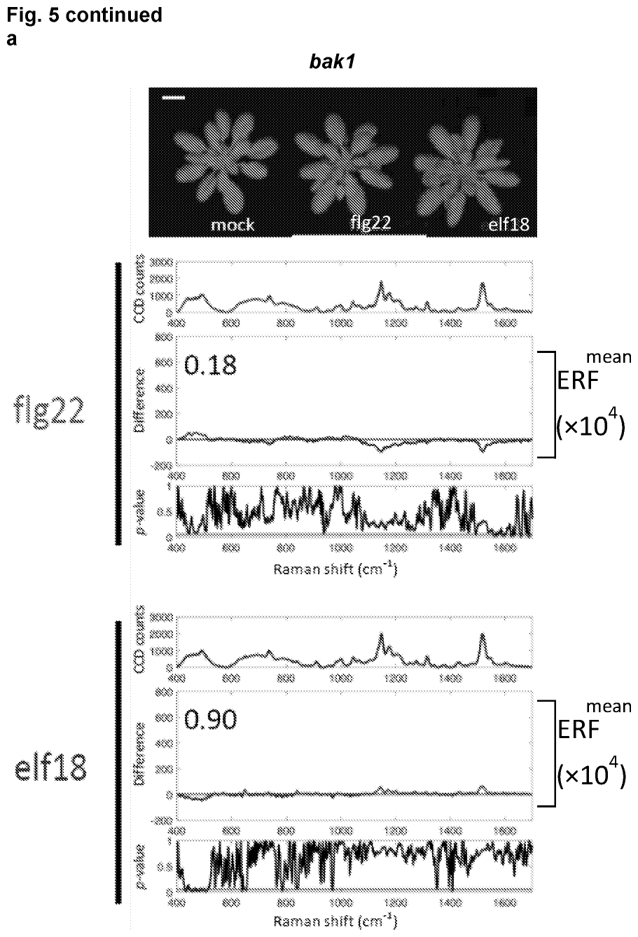

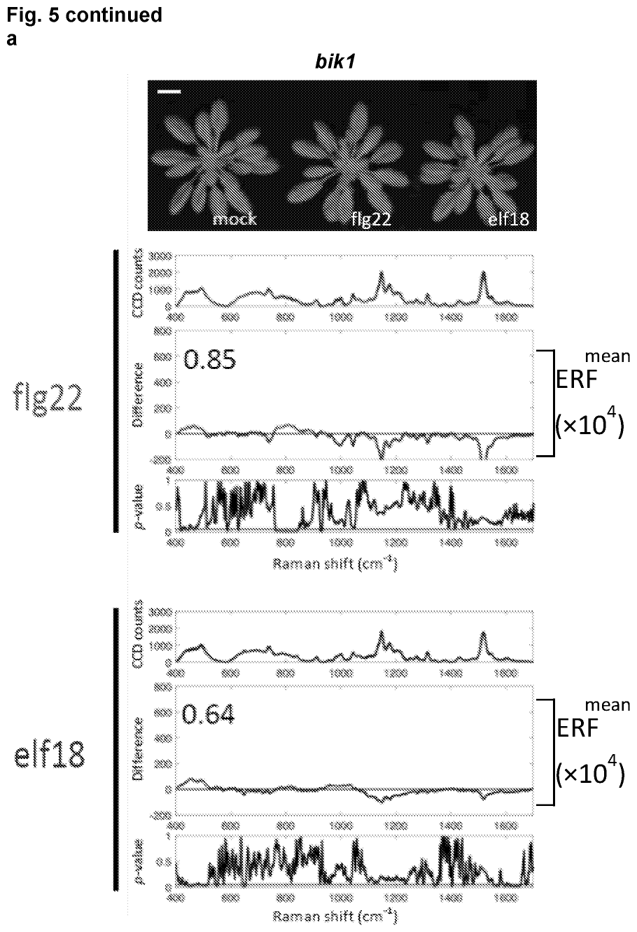

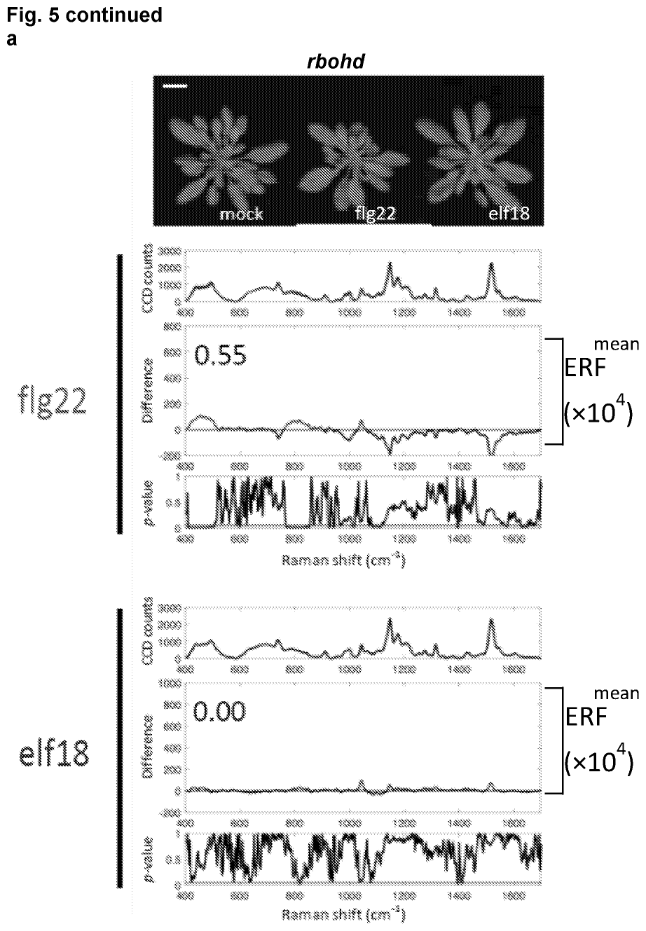

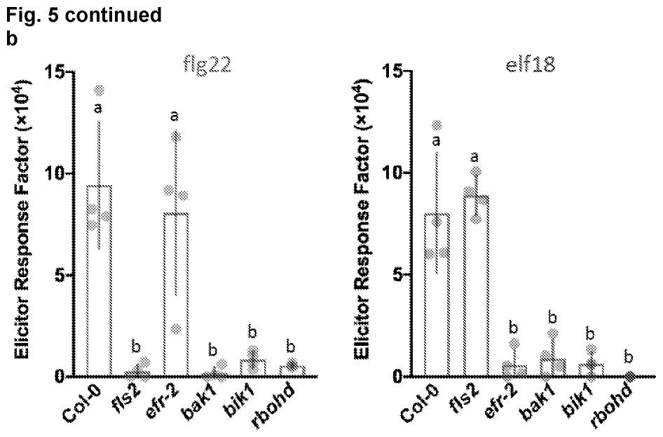

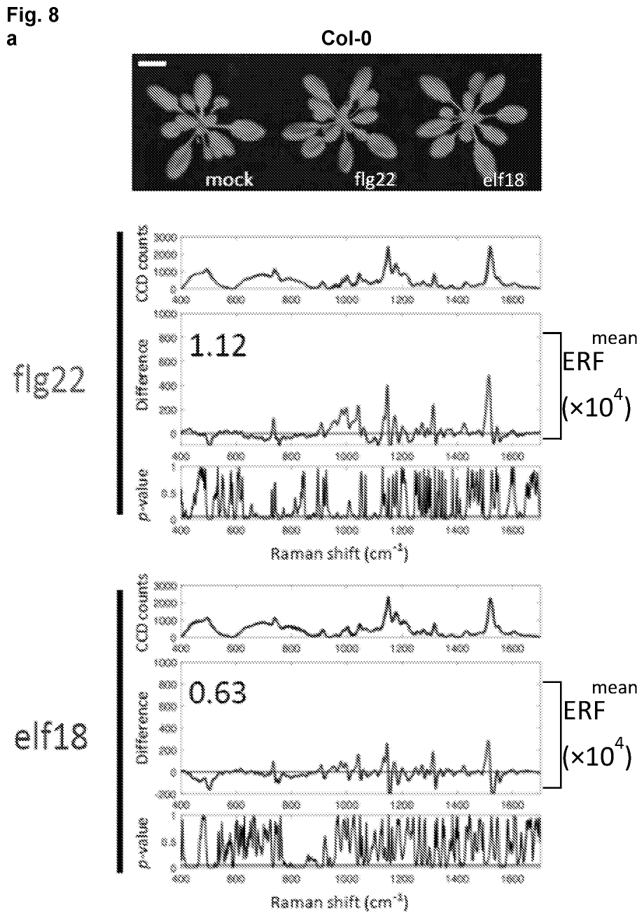

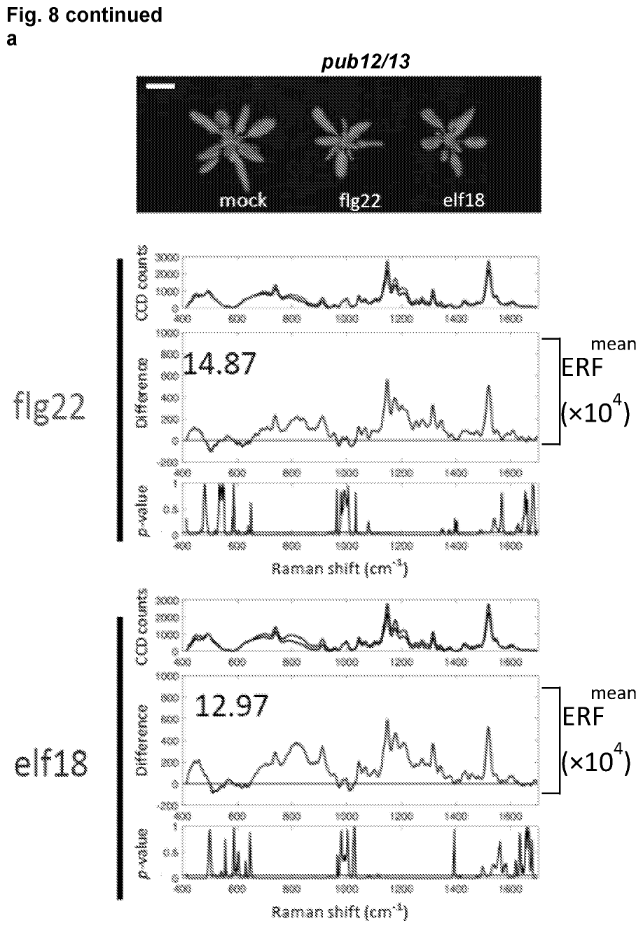

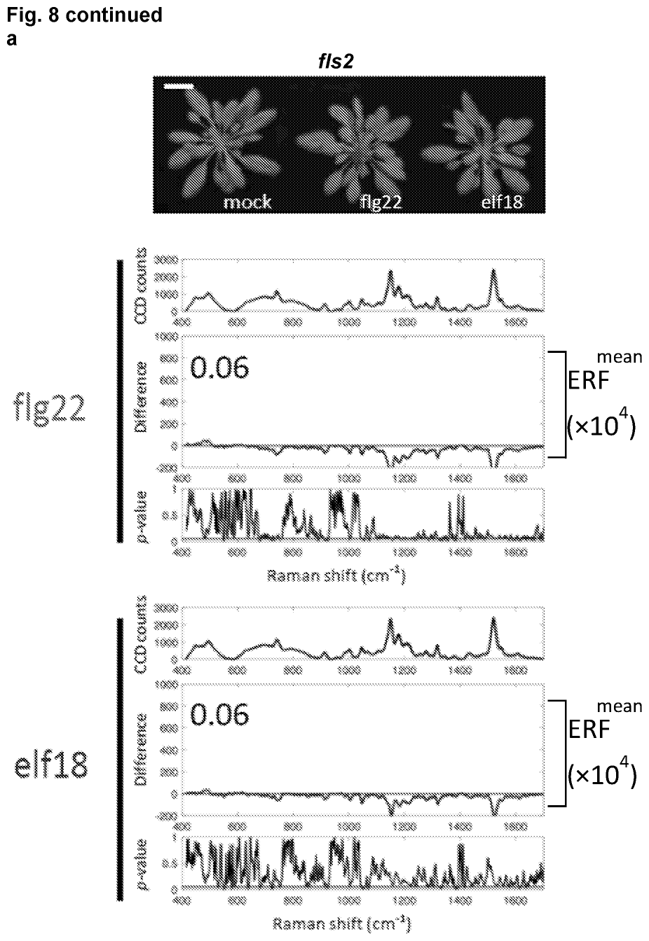

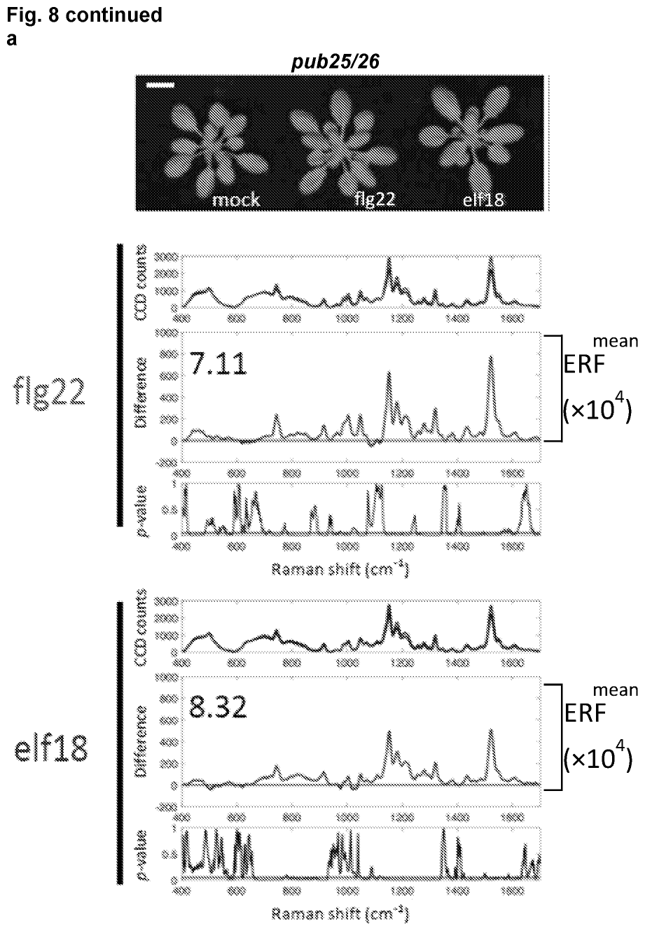

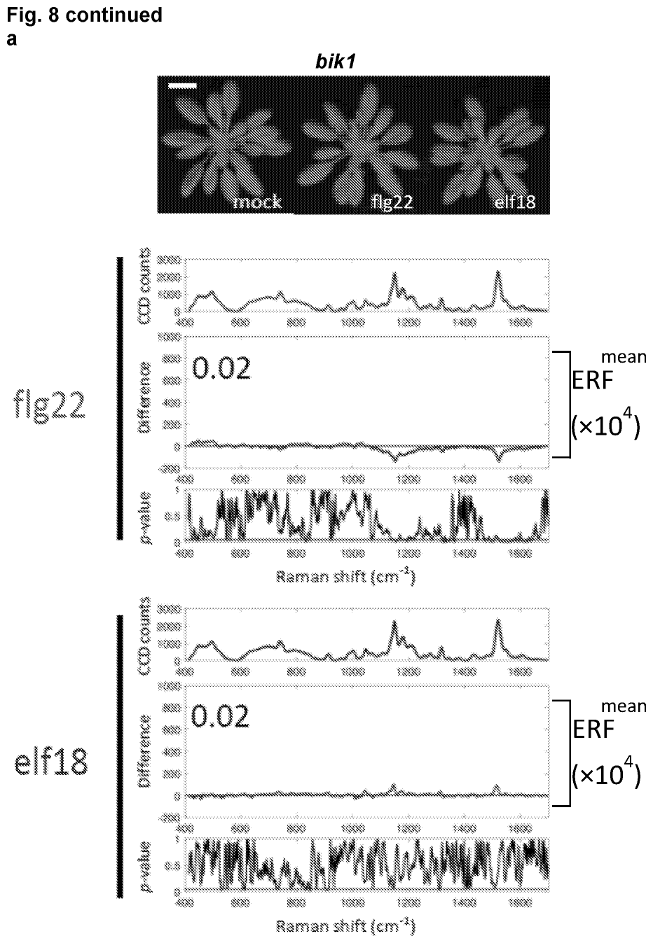

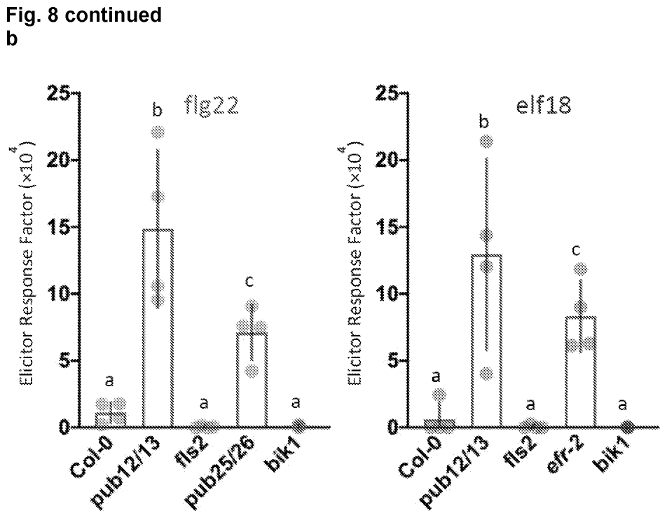

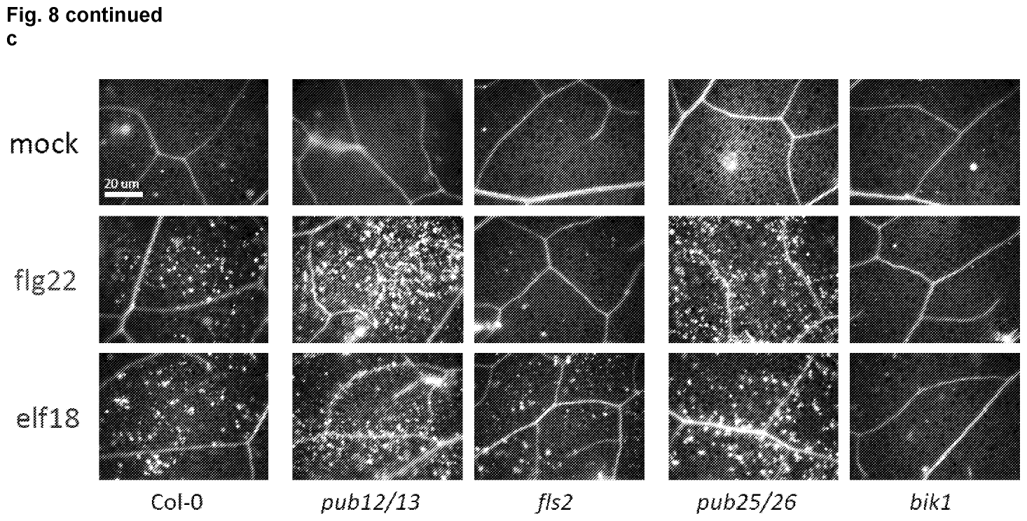

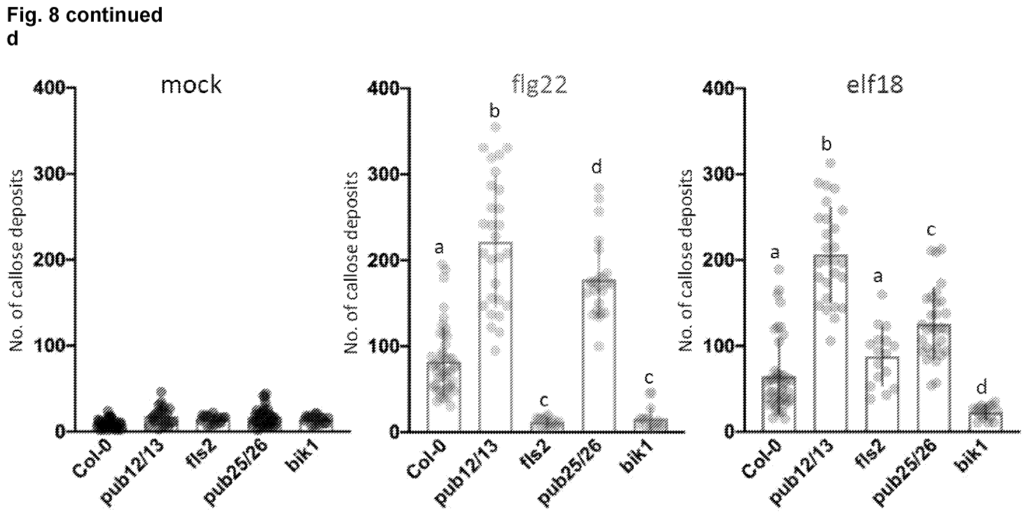

The difference Raman spectrum between the elicitor-treated leaf and the mock-treated leaf shows clear and reproducible changes in Raman shift from about 800 to 1,600 cm⁻¹. These changes are remarkably similar in plants of various genotypes when a positive PTI is expected. However, to assess the statistical significance of the difference spectrum, we performed a t-test and generated a new graph representing the p-value for each Raman shift value. A comparison of the two graphs allows us to focus on the Raman shifts value with a meaningful p-value (p<0.05). Using these data and employing a simple mathematical formula we arrived at the Elicitor Response Factor (ERF) which provides a quantitative measure of the degree of PTI as determined by the difference Raman spectrum (FIG. 4). Our results showed that upon treatment with 1 μM flg22 the ERF value at 10 hpi was 1.12×10⁴ which increased about 8 times by 24 hpi. Similar results were obtained with elf18 (FIGS. 5a,b; FIGS. 8a-d).

The physiological significance of the difference Raman spectrum and the ERF values obtained with WT plants is further strengthened by analysis with several Arabidopsis mutants deficient in PRRs and downstream signaling components. (1) FLS2 and EFR show exquisite specificity with respect to its cognate ligand. Although fls2 is refractory to flg22 it still responds to elf18 treatment and vice versa. (2) The three PTI mutants tested, bak1, bik1, and rbohd, are all non-responsive to either elicitor consistent with the fact that they encode common signaling proteins downstream of the two receptors. (3) Mutants (pub12/13; pub25/26) deficient in negative regulatory components of the PTI pathway (Lu et al., 2011) show Raman spectrum changes at 10 hpi when little changes are detected in WT. These mutants also display early induction of PTI-related genes.

The physiological significance of the difference Raman spectrum and the ERF values obtained with WT plants is further strengthened by analysis with several Arabidopsis mutants deficient in PRRs and downstream signaling components. 1. FLS2 and EFR show exquisite specificity with respect to its cognate ligand. Although fls2 is refractory to flg22 it still responds to elf18 treatment and vice versa. 2. The three PTI mutants tested, bak1, bik1, and rbohd, are all non-responsive to either elicitor consistent with the fact that they encode common signaling proteins downstream of the two receptors. 3. Mutants (pub12/13; pub25/26) deficient in negative regulatory components of the PTI pathway (Lu et al., 2011) show Raman spectrum changes at 10 hpi when little changes are detected in WT. These mutants also display early induction of PTI-related genes,

Examination of the difference Raman spectrum obtained from elicitor/pathogen studies uncovered significant increase in peak intensity in spectral regions as denoted in Tables 2-5. The changes in Raman peaks contributing towards the high ERF or high IRF value at 24 hrs with both the elicitor and pathogens are largely identical indicating that metabolome changes triggered by elicitors and pathogen are similar. The difference in the magnitude of the IRF factor between Arabidopsis and Choysum can be expected because different pathogens can trigger varied immune responses

The most significant increase in peak intensity under all types of elicitor/pathogen treatment was observed at in the following spectral regions; 1,001 cm⁻¹, 1,151 cm⁻¹ and 1,521 cm⁻¹ which corresponds to the characteristic bands attributed to carotenoids (Baranski et al., 2005; Schulz et al., 2005; Lorenz et al., 2017).

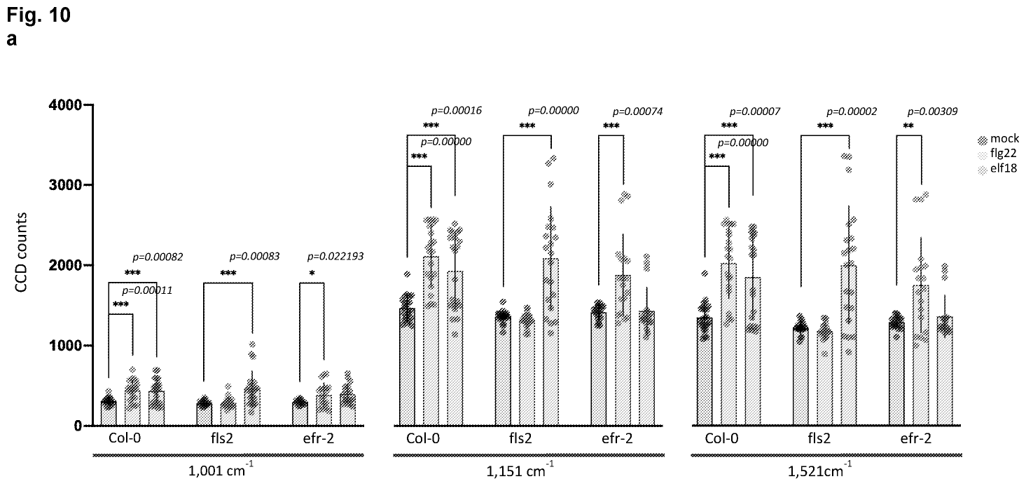

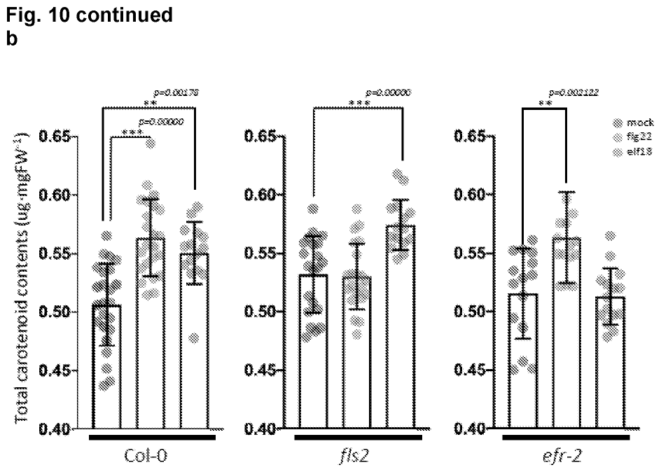

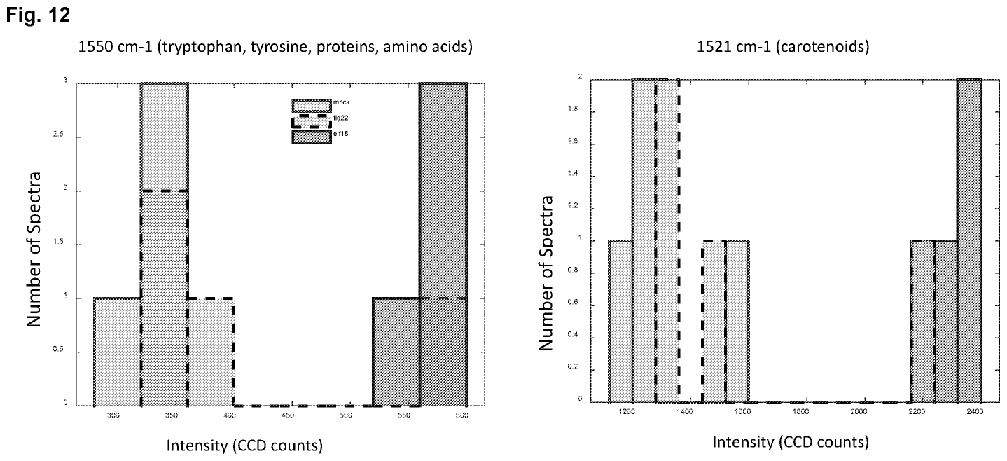

These results suggest that there is a transient increase in carotenoid upon elicitor treatment or infection. To obtain additional evidence, we extracted total carotenoids in control and treated leaves and compared their concentrations in the extract. We found that indeed there was an increase of 5-10% of total carotenoids as a result of elicitor treatment (FIG. 10b). This carotenoid increase is preceded by an earlier increase in the expression of genes in the carotenoid biosynthetic pathway (FIGS. 12a,b). A transient carotenoid increase as an early event of PTI has not been previously reported and its physiological significance remains unclear at this point. Recent research has shown that carotenoids are constantly being synthesized and also degraded to produce other derivatives (Beisel et al., 2010). Apocarotenoids are produced by oxidative cleavage of carotenoids either catalysed by carotenoid cleavage dioxygenases (CCDs) or by non-enzymatic processes. New studies are unravelling the functions of apocarotenoids in plant development and stress response. Important plant apocarotenoids includes phytohormones such as abscisic acid (ABA) and strigolactones and signaling metabolites like β-cyclocitral. (Felemban et al., 2019).

In plants, phytohormone abscisic acid (ABA) (FIGS. 12a,b), plays a role in abiotic/biotic stress response (Jia et al., 2017). Gene analysis showed that expression levels of NCED, which encodes the rate-limiting enzyme for ABA biosynthesis, are highly elevated 3-6 hpi by either elicitor. Expression levels of various CCD genes are also found altered by elicitor treatment, (FIGS. 12a,b). This result is consistent with the notion that the transiently increased carotenoids are subsequently fragmented and modified to produce ABA and other derivatives.

Besides carotenoids, Raman spectral changes implicate transient increase in metabolites like pectin, phenylpropanoids, cellulose, nitrates, lignin, nucleic acid and proteins to be involved in early PTI response. A previous study which evaluated metabolic changes associated with Pseudomonas syringae infection in Arabidopsis at different time points observed changes as early as 8 hpi. The major metabolic pathways perturbed upon infection were phenylpropanoid/lignins, tryptophan/indolic glucosinolates/nicotinic acid, methionine/aliphatic glucosinolates, purine/riboflavin/folate and urea cycle/polyamine/proline metabolic pathways. Other pathways found affected by infection include carotenoid metabolism, chlorophyll degradation and non-aromatic amino acid metabolism. (Ward et al., 2010). The Raman spectral analysis also indicate changes in similar metabolites upon infection in Arabidopsis. Along with carotenoid, the changes between 1549 and 1552 cm⁻¹ region was found to discriminate between the mock and infected plants with high statistical significance. This vibration band is associated with Amide II (C═N and N—H stretch): mainly proteins, tyrosine and tryptophan (Movasaghi et. al. 2007). Ward et al. in their study found that amino acids, tryptophan and tyrosine are significantly increased in abundance by 12 hpi with Pseudomonas syringae in Arabidopsis. Our study also found genes involved in tryptophan metabolism altered by elicitor treatment (FIG. 3). The Raman shift observed at 1549 and 1552 cm⁻¹ under elicitation and infection probably reflects an increase in abundance of tryptophan and tyrosine.

Several groups have used Raman spectroscopy to analyze a number of crop plants infected with bacterial, fungal, and viral pathogens. In most cases, a decrease in carotenoid was consistently seen, which is used as a hallmark to diagnose plant pathogenic diseases. And, we have previously detected a decrease in carotenoid contents in leaf blades and petioles of Arabidopsis with shade avoidance syndrome (SAS) using Raman spectroscopy (Sng et al., 2020). By contrast, we found that a transient increase in carotenoids is an early event in PTI. We note that the carotenoid increase we have detected occurs at about 10-24 hr upon receptor activation before the manifestation of any disease symptoms. By contrast, in most of the previous work, the Raman spectra were taken with plants likely to be several days after pathogen infection as the tissues were already showing disease symptoms. In one study, Raman spectra showed increase in carotenoid peak intensity in pepper plants inoculated with virus at 48hpi. However, total carotenoid content when measured by spectrophotometric methods was found to be unaltered. It was mainly concluded that Raman spectroscopy and spectrophotometric technique might detect different sets of carotenoids (Rys et al., 2014). Plant diseases are known to be a major factor compromising crop yield worldwide (Savary et al., 2019; Velásquez et al., 2018) and early detection of pathogen infection can greatly facilitate disease management. In the minimally invasive experiments described here, leaf discs were excised from the leaf region proximal to the infiltration sites for investigation using Raman spectroscopy and to determine ERF values. However, the availability of a high throughput custom-made portable or hand-held Raman spectrometer (Farber & Kurouski, 2018; Krimmer et al., 2019; Sanchez et al., 2019) would allow Raman spectral analysis to be performed directly with field grown crops in a non-invasive manner, and our group is currently developing such systems. The use of ERF as a diagnostic Raman signature for early PTI will provide an important tool for the identification of plants with early pathogen infection and facilitate effective disease management.

### EXAMPLES

The present invention is described by reference to the following Examples, which are offered by way of illustration and are not intended to limit the invention in any manner. Standard techniques well known in the art or the techniques specifically described below were utilized.

### Example 1

### Materials and Methods

Plant growth and elicitor/pathogen infiltration: Arabidopsis thaliana (Col-0) wild-type (WT) and mutants including fls2 (salk_062054), efr-2 (salk_068675), bak1 (salk_116202), bik1 (salk_005291), rbohd (salk_083046), pub12/13 (Zhou et al., 2018) and pub25/26 (Wang et al., 2018) were used. Ten-day old seedlings grown on soil were transferred to a growth room at 70% humidity and 21° C. with a 10-h-light/14-h-dark photoperiod (short-day condition). Light intensity was 100 μmol/m²s⁻¹. Five to 6 week-old plants with about 17-18 leaves were used. Flg22 contains the 22-amino acid sequence QRLSTGSRINSAKDDAAGLQIA (SEQ ID NO: 1) corresponding to a conserved motif of the amino terminus of flagellin from Pseudomonas species. Elf18 contains the sequence AcSKEKFERTKPHVNVGTIG (SEQ ID NO: 2) corresponding to the conserved N-terminal 18-amino acid peptide of the bacterial protein elongation factor Tu (EF-Tu). These peptides (Axil Scientific Pte Ltd) were dissolved in sterile H₂O to a concentration of 5 mM and aliquots of 100 μM stock solution were stored at −20° C. 3-hydroxydecanoic acid (Sigma, H3648) (Kutschera et al., 2019) was dissolved in ethanol to a concentration of 5 mM, and Chitin, GlcNacβ1-4 [GlcNacβ1-4GlcNAc]5GlcNAc (ELICITYL, GLU437) was dissolved in water to a concentration of 5 mM. Aliquots of 100 μM stock solutions were stored at −20° C.

An overnight culture of Pseudomonas syringae pv. Tomato DC3000 (Pst DC3000) (Rufián et al., 2019) was suspended in 10 mM MgCl₂ to approximately 5×10⁵ cfu/ml (OD₆₀₀=0.001). 50 ul of either the bacterial suspension or 10 mM MgCl₂ (mock) was infiltrated on the abaxial side of Arabidopsis leaf by a needleless syringe. Commercial Choysum (Brassica rapa v. parachinensis) seed were grown in growth chambers at 25° C. under 16-h-ligh/8-h-dark photoperiod with 75% humidity. Two-week old Choysum plants were inoculated with the Xanthomonas campestris py. campestris (Xcc) isolate (ATCC33913). The Xcc strain was grown in YGC (yeast extract, glucose and calcium carbonate) medium at 28° C. Harvested bacterial cells were resuspended in YGC medium to 1×10⁸ cfu/ml. Using a needleless syringe, the abaxial side of the fourth leaf of each plant was inoculated with 200 ul of Xcc suspension on both sides of the central leaf vein near the leaf margins. Leaves of separate plants inoculated with 200 ul of sterile media served as the mock control.

Custom-built Raman spectroscopy system and spectral data analysis: The Raman spectroscopy system with an 830 nm excitation wavelength was previously described (Huang et al., 2020). The Raman shift axis was calibrated using a polystyrene sample (Creely et al., 2005) and Raman spectra were recorded in the range of 400 cm⁻¹-1700 cm⁻¹. The 830 nm excitation wavelength has the advantage of deeper penetration depth and generating comparatively lower fluorescence background signal from the leaf tissue, resulting in spectra with a higher signal to noise ratio (SNR). The pre-processing of the Raman spectra consisted of cosmic ray removal and smoothing with the Savitzky-Golay filter function (MATLAB Inc., USA) with 5th order polynomial and frame length of 11. The Raman spectra presented here were obtained after removing any residual fluorescence background using a polynomial subtraction method with non-negative constraints (Lieber & Mahadevan-Jansen, 2003).

For Arabidopsis, two plants from each independent biological sample were investigated (Huang et al., 2020). The 7^{th} or 8^{th} leaf of 5-6 week-old plants were used for infiltrations (FIG. 1) At the designated time after infiltrations, leaf discs (6 mm diameter) were excised with a puncher from regions proximal to the infiltrated sites (FIG. 2a). Two leaf discs were obtained from each elicitor-treated leaf, one from the left and the other from the right side of the central leaf vein. A total of 4 leaf discs were analyzed for each genotype per experiment. The leaf discs were placed with a drop of water between a fused silica slide of thickness 0.5 mm and a fused silica coverslip, which was 100 μm thick. Three locations were measured in each leaf disc, with 5 Raman spectra acquired per location and an exposure time of 10s per spectrum. Thus, an average Raman spectrum of an independent biological sample was obtained from the mean of total 60 Raman spectra. The experiments were repeated with 3-4 independent biological samples.

For Raman spectral acquisitions in Choysum, two leaf discs from a single leaf were excised from the proximal regions of the infiltrated site at 24 and 48 hrs after infiltration. Three locations were measured in each leaf disc, with 5 Raman spectra acquired per location and an exposure time of 10s per spectrum. Thus, an average Raman spectrum of one independent biological sample for each time point was obtained from the mean of total 30 Raman spectra. Three biological replicates for each time point was performed for both Xcc and mock infiltration.

RNA extraction and gene expression analysis by qRT-PCR: Total RNA was extracted from the entire infiltrated leaf using RNeasy plant mini kit (74104; Qiagen) with DNase I (79254; Qiagen) treatment. cDNA synthesis and RT-PCR were performed as described (Park et al., 2019). Expression of the Arabidopsis Act2 (At3g18780) was used as an internal control. Primer pairs for quantitative RT-PCR were designed using Primer-BLAST (https://www.ncbi.nlm.nih.gov/tools/primer-blast/index.cgi) or manually by eye. All RT-PCR primers are listed in Table 1.

TABLE 1
Primer Sets for qRT-PCR

| Gene ID | Name | FORWARD (SEQ ID NO:) | REVERSE (SEQ ID NO:) |
|---|---|---|---|
| At3g18780 | ACT | TCCCTCAGCACATTCCAGCAGAT (3) | AACGATTCCTGGACCTGCCTCATC (4) |
| At2g14610 | PR1 | ACGGGGAAAACTTAGCCTGG (5) | TTGGCACATCCGAGTCTCAC (6) |
| At5g44420 | PDF1.2 | GGTTGGAATATGGTAGTGCTGT (7) | CTGAACGGATGAAGGTGGAA (8) |
| At3g45640 | MPK3 | CAACTTCCCACGTCAGCCCT (9) | CGGGGCTGCTGCACTTCTAA (10) |
| At4g23550 | WRKY29 | CGGAGATGGAGACAAGTGGCTT | TGTGAGGATCGTTTGTGTGGAGAA |
| | | (11) | (12) |
| At5g24110 | WRKY30 | CCCTTTGGAATCCCCGTTTG (13) | GAATAGAACCCACCAAATCCTCCA |
| | | | (14) |
| At4g23810 | WRKY53 | ACACCACCATTAGCCTCGCC (15) | ACGCGGGGAAAGTTGTGTCA (16) |
| At5g59820 | ZAT12 | ACGGTGGCGATCAAAAACGC (17) | TGGGACAAGGATGCGACGAC (18) |
| At2g28390 | SAND | GCTGGTGCACAGAAAGCGTC (19) | CCTCAAGGCGTACCCTGCAA (20) |
| At1g74080 | MYB122 | ACCTCTTCGAATCTCCCCATC (21) | AACTTCATTGATCGGCGTCAC (22) |
| At1g18570 | MYB51 | CTACAAGTGTTTCCGTTGACTCTGAA | ACGAAATTATCGCAGTACATTAGAGG |
| | | (23) | A (24) |
| At5g60890 | MYB34 | CACGACTGTCGATAATTTTGGGTT | CATATTGTCATCTTCGTTCCAGGA (26) |
| | | (25) | |
| At4g39950 | CYP79B2 | AACAAAAAGAAACCGTATCTGCCAC | TCCTAACTTCACGCATGCTATCTC (28) |
| | | (27) | |
| At2g22330 | CYP79B3 | CTCCTTCTTCCTTGCAAATGGA (29) | GAGAATCATCAAGAAGCAAAGGG |
| | | | (30) |
| At4g31500 | CYP83B1 | GGCAACAAACCATGTCGTATCAAG | CGTTGACACTCTTCTTCTCTAACCG |
| | | (31) | (32) |
| At2g30770 | CYP71A13 | AATCTGAACTACATCCCATTCGGA | TTAGATCAGGTTGATCCCCATTTG |
| | | (33) | (34) |
| At3g26830 | CYP71B15 | CGTTTGGATCGTGTAGGAGAATATG | TGTTCCCCATTGATGTCTTTG (36) |
| | (PAD3) | T (35) | |
| At4g03550 | PMR4 | CTGGAATGCTGTTGTCTCTGTTG | TCGCCTTTTGATTTCTTCCCAGT (38) |
| | | (37) | |
| At5g17230 | PSY | GACACCCGAAAGGCGAAAGG (39) | CAGCGAGAGCAGCATCAAGC (40) |
| At4g14210 | PDS | GTCGGTCACGCGCTCAGGTA (41) | CGAGATGCTGACATGGCCAGA (42) |
| At1g10830 | Z-ISO | TTGGGGCGTGGAATGGAGACAG | CATAGTGGTTACAGAGTGTTGAGCC |
| | | (43) | 44) |
| At3g04870 | ZDS | GAAGTCACTTGGTCGTCGGTTG (45) | TGCTTCTTCTTCATGTACCCAT (46) |
| At1g06820 | CRTISO | GAAAAGGAAGGGATCCTTGG (47) | GCAAAGCATTGACTGTGCTC (48) |
| At3g10230 | βLCY | CTTGGTTCAGTATGACAAACCTTAC | GACCAGGTCTAGCAACTAAAGAAGT |
| | | (49) | (50) |
| At5g57030 | ΕLCY | TCGGTTTGTGTAGTGTCAGAGC (51) | AAGGTCAAACCAGCAGGACC (52) |
| | (LUT2) | | |
| At1g31800 | CYP97A3 | GGACATGAAACATCGGCGGCAG | GAGGAGCTCCTGGTGCAATCTG (54) |
| | (LUT5) | (53) | |
| At3g53130 | CYP97C1 | GCACGGAAGCTTCGAAAAGAGG | CCGAGGAGATCAGCGACATCG (56) |
| | (LUT1) | (55) | |
| At4g15110 | CYP97B3 | CTGTGCTTGGTCAAGGTCCACC (57) | AGATGGATCAAAGCCAGCCC (58) |
| At4g25700 | CHYB | CTTGGCAAATGGAGGGAGGTG (59) | CGTTATGCCTAACCCGGCG (60) |
| | (BCH1) | | |
| At5g52570 | CHYB2 | GTTGGGATGGAGTTTTGGGCAAG | CTGTGTGGTGTAGCTGGTGAGC (62) |
| | (BCH2) | (61) | |
| At1g12520 | CCS | CCATGCCTCAGCTTCTTACTGAG | GTACAAGCTCCCTGTGCTGGC (64) |
| | | (63) | |
| At5g67030 | ZEP | TGCAATGGCGAGGATGGCTG (65) | ATGCATCTTCGAAACCTGAGACG (66) |
| | (ABA1) | | |
| At1g08550 | NPQ | TGCCAGCTGCATGAGTTCCAC (67) | AATTCTCTCCACGAGCGCAGG (68) |
| At1g67080 | NXS | TCCGGTGTACGAGCTTCATGG (69) | CCAGACAACTCTGGCAAC (70) |
| | (ABA4) | | |
| At3g14440 | NCED3 | CCGAGCCGTGGCCTAAAGTC (71) | CACGACCTGCTTCGCCAAATC (72) |
| At1g52340 | ABA2 | TAGGAGTGTTGCAGCGGAGC (73) | ATCAACCGTCAGTTCCACCCC (74) |
| At2g27150 | AA03 | TAGCACGACATCCGAGAGCAG (75) | AAGGTCCACTTCCACCTCGC (76) |
| At1g64680 | D27 | GAGCTTCGTGGAGGTGTCAAAG | GGTTCATGGTGAGTGGGAGG (78) |
| | | (77) | |
| At3g63520 | CCD1 | CCGCATCTGCGGTTGATTTCCC (79) | CCATATGGGACCCTGTGCGG (80) |
| At4g19170 | CCD4 | AGTACGCCGGAGATGAGTCG (81) | CGAAAAACGGTTCTCCGCCG (82) |
| At2g44990 | CCD7 | CCTCTAAACGGGTGGAACAAGCC | CCTCGGAACCTCTAACCTCGAC (84) |
| | | (83) | |
| At4g32810 | CCD8 | GGCTAGCGTGGAGGTTCCAG (85) | CCACAATATCAACCTTGGAGAGAGC |
| | | | (86) |
| At2g26170 | CYP711A | ATTGACGGGTTTGGGAACCGTG | CAGGCTCGTGGACCGATACC (88) |
| | | (87) | |

Elicitor-induced callose deposition: Callose assay was performed as described (Ton et al., 2005). The callose-mediated fluorescence was visualized using a DAPI filter set (excitation filter 365 nm; dichroic mirror 395 nm; emission filter 397 nm) of an Axio Imager M2 microscope (Zeiss, Germany). Images collected with a photometric CoolSNAP HQ2 camera system (Roper Scientific Germany) were acquired with a 100× lens using MetaMorph software (Version 7.7.0.0; Molecular Devices, Sunnyvale, CA). Callose foci within the frame of a single image (magnification ×100; 5,655 μm² per region) were quantified using the ImageJ software (http://rsb.info.nih.gov/ij/, Schneider et al., 2012). For each experiment, the number of callose deposits was measured in 2 plants with about 4-5 leaf regions surveyed per plant. This gave about 8-10 data points per experiment. The experiment was repeated with 3 independent biological samples. For Choysum, callose deposition was visualized in 3 independent biological samples at 24 and 48 hpi.

Total carotenoid extraction for spectrophotometric analysis: Total carotenoids were extracted and analyzed as described with minor modifications (Sumanta et al., 2014). Briefly, 30 mg of fresh leaf sample was homogenized in a tissue homogenizer with 600 ul of methanol solvent. After centrifugation at 10,000×g for 15 min at 4° C., 0.1 ml of the supernatant was mixed with 0.9 ml of methanol. The mixture was analyzed for photosynthetic pigment content by a spectrophotometer (TECAN, The spark multimode microplate reader) according to Sumanta et al. (2014). For each experiment, the quantitation of total carotenoids was measured in 3-5 infiltrated plants per experiment. The quantification of carotenoids was expressed as the mean value of 15-20 different leaf extracts in four replicated experiments.

### Example 2

### Flg22 and Elf18 Elicit Robust PTI Response

flg22 and elf18 were used to induce plant innate immunity responses without related pathogenic symptoms (Bektas & Eulgem, 2015). To reduce variability between biological samples, Arabidopsis wild-type (Col-0) and mutant plants were grown under short-day conditions for about 5-6 weeks with about 17-18 leaves (Farmer et al., 2013; Kurenda et al., 2019) (FIG. 1a). The abaxial side of leaf 7^{th} or 8^{th} was infiltrated with a needleless syringe containing (50 ul) water, 1.0 μM flg22 or 1.0 μM elf18 (FIG. 1b). Twenty-four hours post-infiltration (hpi), no pathological symptoms were seen at the infiltration sites in response to the PAMP nor the slight wounding caused by the syringe infiltration (FIG. 1c). However, compared to mock infiltration, infiltration of either elicitor induced expression of PTI-specific marker genes of the PTI pathway but with different kinetics (FIGS. 3a-3c). Some early responsive genes (WRKY30, MPK3, MYB51, CYP79B2, etc.) were rapidly induced at 3 hpi whereas expression of other genes (PR1, PDF1.2, ZAT12, and MYB122) was detected only at 6 hr. Expression of most activated genes returned to basal levels by 24 hours (FIGS. 3a-3c) although several genes (PR1, WRYK29, and WRKY53) still maintained high expression levels at this time point. One day after infiltration, callose deposition in the elicitor-infiltrated leaf was significantly induced more than in the mock-infiltrated control leaf (FIG. 1d) consistent with the increased expression of genes related to callose synthesis (FIGS. 3a-3c). The gene expression results and the callose deposition data together confirmed that under our experimental conditions both flg22 and elf18 induced rapid and robust innate immunity response in the infiltrated leaves.

### Example 3

### Analysis of the PTI Response by Raman Spectroscopy: Development of Elicitor Response Factor (ERF)

A custom-built near infrared excitation Raman instrument (FIG. 1e) to measure Raman spectra from mock- or elicitor-infiltrated leaf samples (FIG. 2). To quantify the PTI response using Raman spectroscopy, we developed an elicitor response factor (ERF) as described below (FIGS. 4a-4b):

Step 1: After pre-processing, described in the Materials and Methods section, the mean of 60 Raman spectra from each individual biological sample was obtained in the Raman shift spectral range of 400 cm⁻¹-1,700 cm⁻¹.

Step 2: The difference of the mean spectra, as obtained in Step 1 above, between elicitor-treated and mock control-treated samples were subsequently derived to highlight different Raman spectral regions with positive values.

Step 3: A p-value plot was obtained using a t-test to evaluate the statistical significance of difference Raman spectra obtained in Step 2. Difference Raman spectral regions where p-value <0.05 were noted. The p-value plot was corrected by including an estimation of the positive false discovery rate (pFDR) and applying the multiple-hypothesis testing principle.

Step 4: The area under the curve of the difference Raman spectral region which has a positive value and also represents the corrected p-value <0.05 of Step 3 is defined as the ERF, which we used to measure the level of PTI response. A higher ERF value indicated a higher level of the elicitor-induced immune response.

Step 5: All the positive spectral regions contributing towards ERF were tabulated.

### Example 4

### Effects of Elicitor Concentration on PTI Response Measured by Raman Spectroscopy

Because robust induction of PTI marker genes was seen with 1 μM flg22 or elf18 (Seo et al., 2019) we use this elicitor concentration to perform a time course experiment on leaf regions proximal (PR) or distal (DR) from the infiltrated sites (FIGS. 2a,b,c). For flg22, the highest Raman shift spectrum and a high ERF value were obtained at the PR 24 hpi. The ERF value decreased to almost zero at 48 and 72 hpi (FIGS. 2b,d). Low ERF values were obtained in the DR which gradually increased with time at 48-72 hpi indicating signal transmission from the PR to the DR. For elf18, Raman shift signals were also detected at 24 hpi in the PR with high ERF values which decreased to almost zero at 48-72 hpi. Lower ERF value was seen in the DR (FIGS. 2c,d). Based on these results, the Raman spectra were analyzed at the proximal region (PR) 24 hpi with an elicitor concentration of 1.0 μM for subsequent experiments.

### Example 5

### Raman Spectral Analysis of PAMP-Signaling Mutants Defective in Positive or Negative Regulatory Function

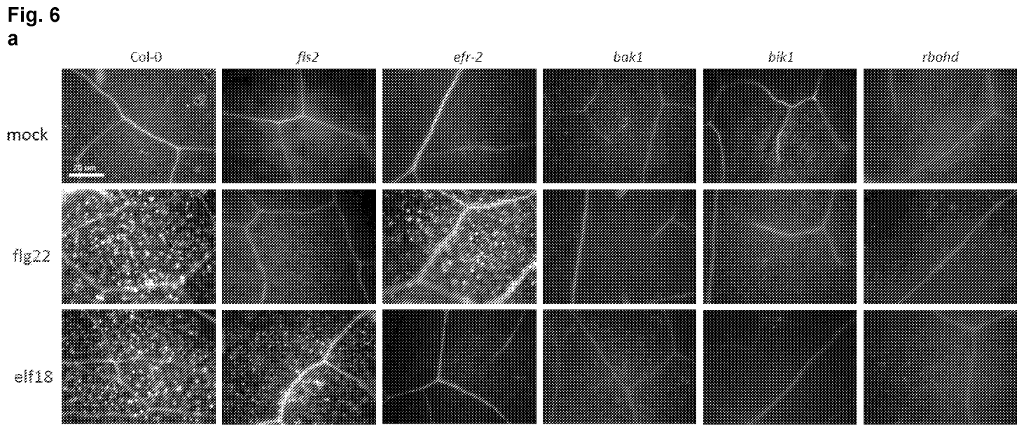

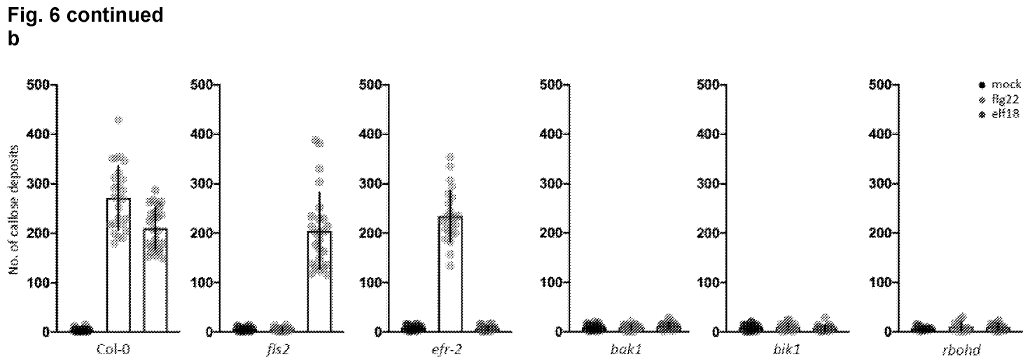

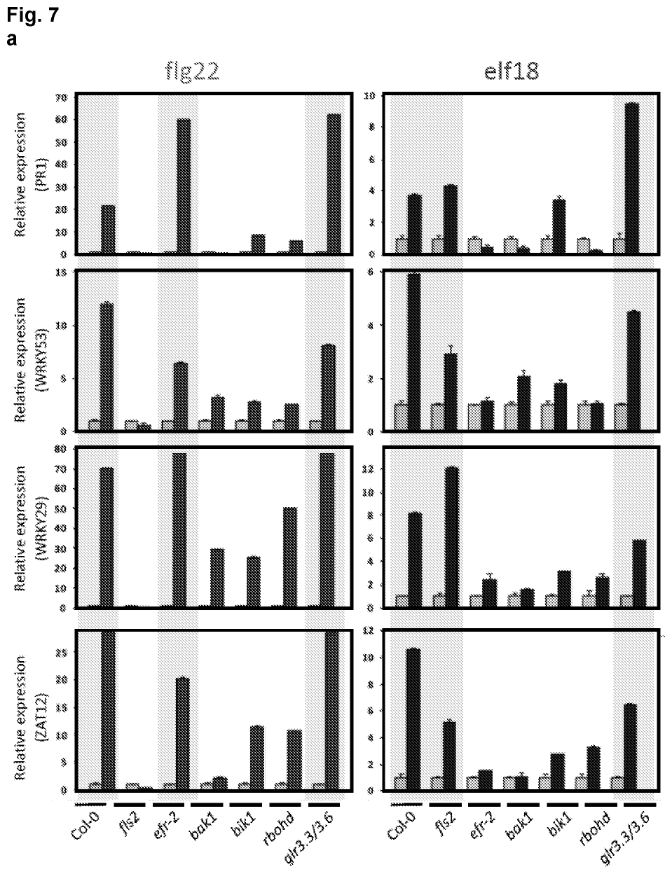

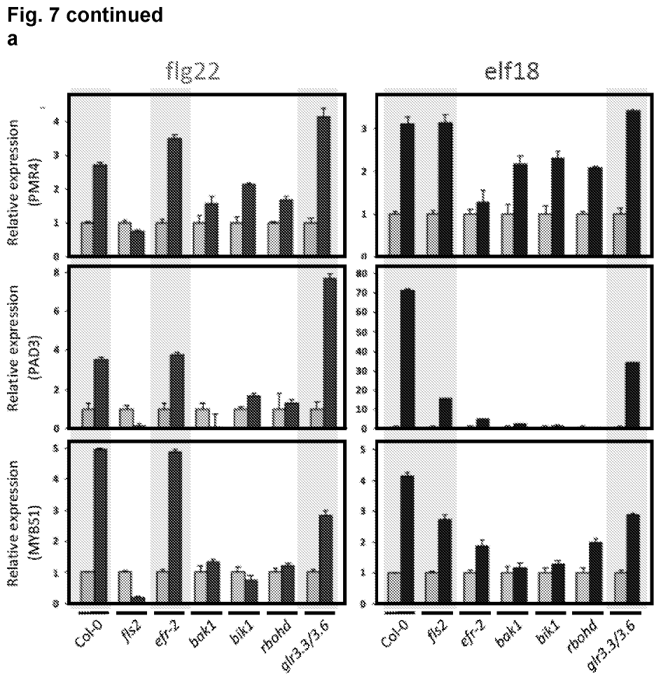

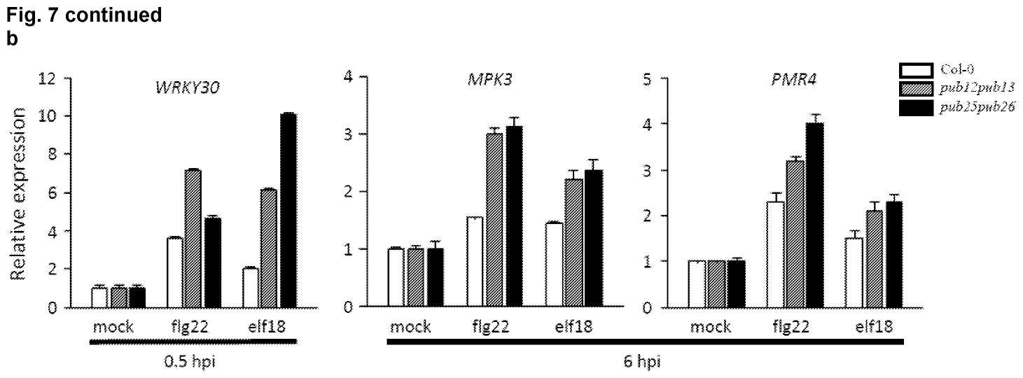

To confirm that the Raman spectra changes obtained with the elicitors were indeed caused by PTI signaling, we first analyzed Raman spectra of a well-characterized mutant of the PTI pathway. Arabidopsis plants possess two PAMP receptors. The FLS2 receptor specifically recognizes flg22 whereas the EFR receptor is specific for elf18 (Chinchilla et al., 2006; Zipfel et al., 2006). FIG. 3 shows that WT plants responded to both flg22 and elf18 with similar ERF values. By contrast, the fls2 mutant was refractory to flg22 but remained responsive to elf18, and similarly, the efr-2 mutant responded to flg22 but not to elf18 (FIGS. 5a,b). These results confirmed that the immune response is specific to the elicitor used. We also measured Raman spectra of mutants including bak1, bik1, and rbohd that are blocked in the PAMP signal transduction downstream of receptor activation (FIGS. 5a,b). Previous publications have reported that the following mutants of the PAMP signaling pathway, fls2, efr-2, bak1, bik1, and rbohd, have significantly reduced callose deposition after elicitor treatment (Lu et al., 2009; Zhang et al., 2010; Jaillais et al., 2011; Belkhadir et al., 2012; Daudi et al., 2012; Chaudhary et al., 2014; Smith et al., 2014). Callose deposition patterns and expression changes of markers genes of these mutants confirmed these previous results and verified their positive regulatory role in PTI response (FIGS. 6a,b; FIG. 7a). All these mutants did not show any significant changes in Raman spectra after elicitor treatment, hence had low ERF values of <1×10⁴ (FIGS. 5a,b).

Following the activation of the PAMP receptor, the PTI signaling pathway is fine-tuned by down-regulation to prevent excessive or prolonged activations that would be deleterious to the host plants. One common mechanism of signaling desensitization is by polyubiquitin-mediated proteolysis of signaling components and several such components have been identified. The FLS2 receptor itself is known to be poly-ubiquitinated by PUB12/13 (Lu et al., 2011; Zhou et al., 2015) and the co-activator BIK1 is similarly modified by PUB25/26 (Wang et al., 2018). Therefore, both PUB12/13 and PUB25/26 are negative regulators of the PTI response, and mutants deficient in these E3 ligases should display hyper-responsiveness to elicitors. In a time-course experiment, we found that the pub mutants (pub12/13 and pub25/26) indeed showed increases in gene expression levels as early as 0.5 hr or 6 hr after 1 μM elicitor treatment when little response was detected with WT (FIG. 7b). Based on the earlier gene expression kinetics we decided to measure changes in Raman spectra at 10 hpi. FIGS. 8a-8d shows that at 10 hr after flg22 treatment, the WT ERF value was only 1.12×10⁴ as compared to an ERF value of 9.44×10⁴ at 24 hr hpi (FIG. 5a). Similarly, in the case of elf18, the ERF value at 10 hpi was 0.63×10⁴ compared to 8.02×10⁴ at 24 hpi (FIGS. 8a,b for 10 hr; FIGS. 5a,b for 24 hr). These results suggest that in WT plants the PAMP signalling pathway was only slightly activated at 10 hr and full activation would require 24 hpi. By contrast, at 10 hpi of either elicitor pub12/13 displayed an ERF value about 13-21 times higher than WT values, and for pub25/26, about 6.4-13.3 times higher (FIG. 8b). These results showed that the hyper-responsive nature of the pub mutants to elicitors can be confirmed by Raman spectroscopy. Interestingly, higher ERF values were found in pub 12/13 targeting a FLS2 receptor compared to those of pub25/26 targeting a downstream regulator, BIK1, in the flg22/elf18 signaling pathway (FIG. 8b).

The ERF values were evaluated based on the Raman spectrum analysis at 24 spots in 4 individual plants (two leaf discs per plant) with 4 independent experiments giving a range of numbers between the high (>5×10⁴) and low (0.0-1.0×10⁴) level according to the PTI response (FIG. 8b). These results confirmed the accuracy of the ERF indexing based on Raman spectral assay for determining PTI responses. Consistent with these analyses, the pub 12/13 and pub25/26 mutant plants showed more callose deposits than WT and the two control mutants, fls2 and bik1 (FIGS. 8c,d)

### Example 6

### Elucidation of Metabolic Changes Contributing Towards the High ERF Values at 24 Hpi

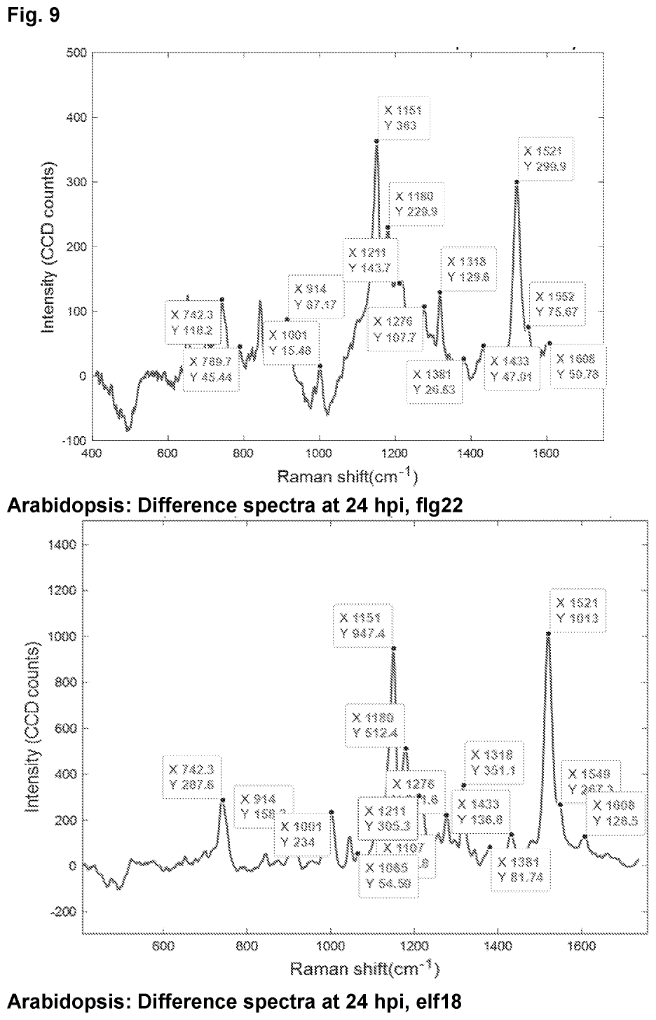

The ERF presents a quantitative and holistic measure of changes in the Raman spectra in elicitor-treated samples relative to mock control samples. Here, we consider changes in the peak intensities for individual vibrational bands and consider the metabolites that give rise to these changes. For WT plants at 24 hpi majority of the Raman peaks that exhibit changes are common to both elicitor response, flg22 and elf18. The difference spectra with annotated peaks are shown in FIG. 9. Tables 2 and 3 summarize the relevant vibrational bands (p<0.05) for the various elicitors, peak assignments are based on previously published reports. Increase in peak intensity at vibrational bands originating from carotenoids, pectin, phenylpropanoids, cellulose, nitrates, lignin and proteins are observed which contributes towards the high ERF value seen at 24hpi.

TABLE 2
Significant Vibrational Bands and Their Assignments that Contribute
to the High ERF Value at 24 hrs Following flg22l Treatment

| Raman peak | | |
|---|---|---|
| (cm−1) | Assignment | Molecular bond |
| 742.3 | Pectin | γ(C—O—H) of COOH |
| | | ring br. mode (aromatics) [##] |
| 789.7* | Chlorophyll a, phosphodiester | (OPO) sym [#] |
| 914 | Leucine, Cellulose, Lignin | (C—C), (C—N) stretching [$] |
| 1001 | Phenylalanine, carotenoids | ν3 (C—CH3 stretching), |
| | | phenylalanine ring stretching mode [##] |
| 1107 | Leucine, Carbohydrates | (C—C), (C—N) stretching |
| | | (C—OH), (C—O—H) stretching [$] |
| 1151 | Carotenoid | C—C stretching; v(C—O—C) [##] |
| 1180 | Carotenoid | dCH, u (pyr half-ring)ₐₛ [$$] |
| 1211 | Phenylalanine, tryptophan | u (C—C₆H₅), u₁₈ (dCₘH) [$$] |
| 1276 | Carotenoid | Amide III [#] |
| 1318 | Cellulose/Lignin/Protein | dCH2 bending [##] |
| 1381 | Proteins, nucleic acids | dCH3 symmetric [#] |
| 1433 | Proteins, lipids | CH₂ scissoring [#] |
| 1521 | Carotenoid | —C═C— (in-plane) [#] |
| 1552 | Amide II, Proteins: tyrosine, | Tryptophan, u (C═C) [#] |
| | tryptophan, Chlorophyll a | |
| 1608 | Lignin | u (C—C) aromatic ring + s(CH) [##] |

*Denotes vibrational bands which were specific to each elicitor.
[#] Movasaghi, Zanyar, Rehman, Shazza and Rehman, Ihtesham U. (2007), Applied Spectroscopy Reviews, 42: 5, 493-541
[##] Shilpi Gupta, Chung Hao Huang, Gajendra Pratap Singh, Bongsoo Park, Nam Hai Chua, Rajeev Ram. (2020), Scientific Reports, 10, 20206.
[$] Birech Z, Mwangi P W, Bukachi F, Mandela K M (2017). PLoS ONE 12(9): e0185130
[$$] Perez-Guaita, David & Kochan, Kamila & Martin, Miguela & Andrew, Dean & Heraud, Philip & Richards, Jack & Wood, Bayden. (2016).. Vibrational Spectroscopy. 91. 10.1016/j.vibspec.2016.07.017.

TABLE 3
Significant Vibrational Bands and Their Assignments that Contribute
to the High ERF Value at 24 hrs Following elf18 Treatment

| Raman | | |
|---|---|---|
| peak | | |
| (cm−1) | Assignment | Molecular bond |
| 742.3 | Pectin | γ(C—O—H) of COOH |
| | | ring br. mode (aromatics) [##] |
| 914 | Leucine, Cellulose, Lignin | (C—C), (C—N) stretching [$] |
| 1001 | Phenylalanine, carotenoids | ν3 (C—CH3 stretching), |
| | | phenylalanine ring stretching |
| | | mode [##] |
| 1065* | Lipids | Skeletal C—C stretch of lipids |
| | | [#] |
| 1107 | Leucine, Carbohydrates | (C—C), (C—N) stretching |
| | | (C—OH), (C—O—H) |
| | | stretching [$] |
| 1151 | Carotenoid | C—C stretching; v(C—O—C) |
| | | [##] |
| 1180 | Carotenoid | dCH, u (pyr half-ring)ₐₛ [$$] |
| 1211 | Phenylalanine, tryptophan | u (C—C₆H₅), u₁₈ (dCₘH) [$$] |
| 1276 | Carotenoid | Amide III [#] |
| 1318 | Cellulose/Lignin/Protein | dCH2 bending [##] |
| 1381 | Proteins, nucleic acids | dCH3 symmetric [#] |
| 1433 | Proteins, lipids | CH₂ scissoring [#] |
| 1521 | Carotenoid | —C═C— (in-plane) [#] |
| 1549 | Amide II, Proteins: | Tryptophan, u (C═C) [#] |
| | tyrosine, tryptophan, | |
| | Chlorophyll a | |
| 1608 | Lignin | u (C—C) aromatic ring + s(CH) |
| | | [##] |

*Denotes vibrational bands which were specific to each elicitor.
[#] Movasaghi, Zanyar, Rehman, Shazza and Rehman, Ihtesham U. (2007), Applied Spectroscopy Reviews, 42: 5, 493-541
[##] Shilpi Gupta, Chung Hao Huang, Gajendra Pratap Singh, Bongsoo Park, Nam Hai Chua, Rajeev Ram. (2020), Scientific Reports, 10, 20206.
[$] Birech Z, Mwangi P W, Bukachi F, Mandela K M (2017). PLoS ONE 12(9): e0185130
[$$] Perez-Guaita, David & Kochan, Kamila & Martin, Miguela & Andrew, Dean & Heraud, Philip & Richards, Jack & Wood, Bayden. (2016). Vibrational Spectroscopy. 91. 10.1016/j.vibspec.2016.07.017.

Among these, the most prominent increase in peak intensity was associated with carotenoids. WT plants showed a significant increase in peak intensities at around 1,001, 1,151 and 1,521 cm⁻¹ at 24 hpi for both the elicitors (FIG. 10a). Peak intensity at 1,151 and 1,521 cm⁻¹ was also increased in the case of receptor mutants treated with heterologous elicitor (FIG. 10a). These strong Raman peaks are attributed to carotenoids and it is these molecules that exhibit the largest change in concentration. Previous reports have attributed two peaks in the 1,100-1,200 and 1,400-1,600 cm⁻¹ Raman shift regions to carotenoids (Payne and Kurouski, 2021). These peaks are due to in phase carbon-carbon stretching vibrations of the main polyene chain with the exact Raman shift locations of these peaks being dependent in the polyene chain length. These results suggest that an early event in plant PTI response is likely an increase in carotenoid concentration which contributes significantly towards the high ERF value.

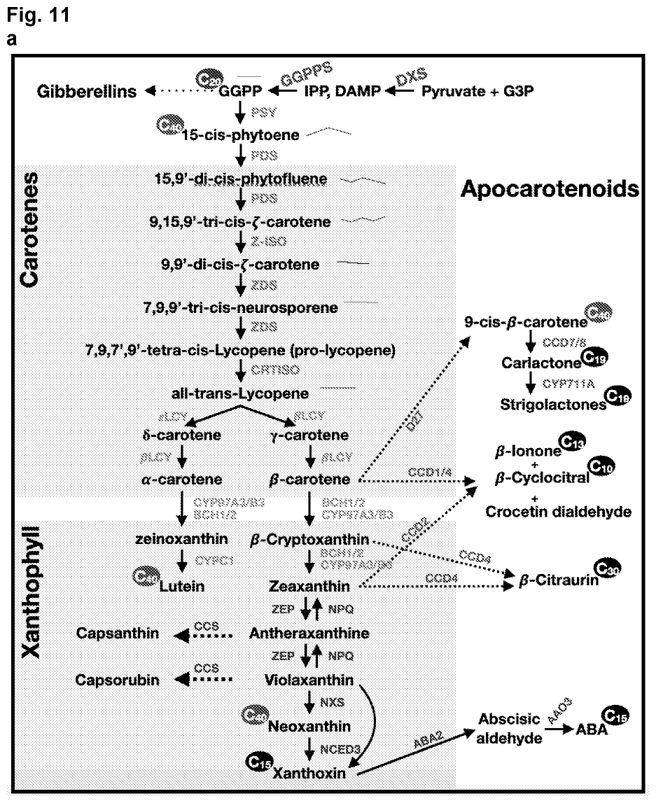

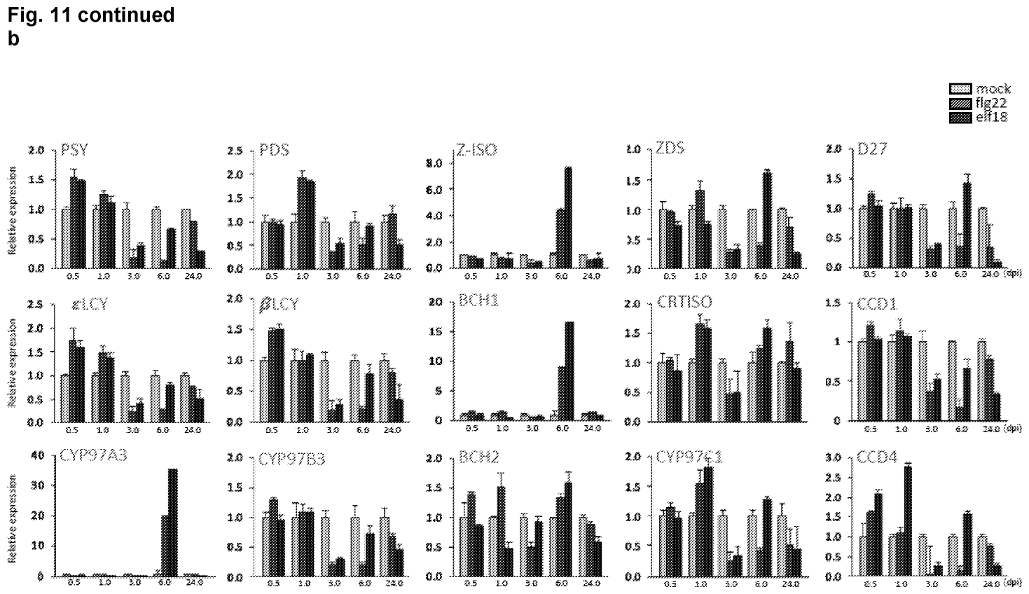

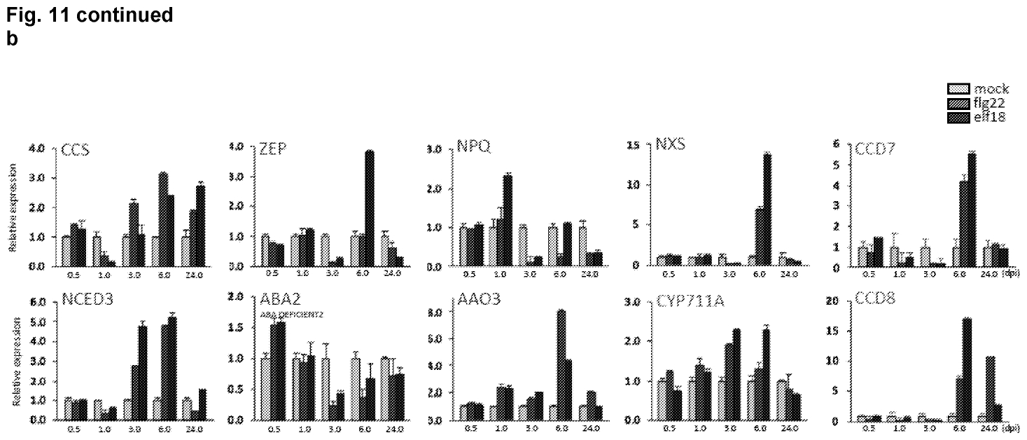

To confirm the Raman spectral results with regards to increase in carotenoid concentration, we extracted photosynthetic pigments (chlorophyll a, b, and carotenoids) from treated leaves and measured changes in their content directly. Treatment of WT with elicitors produced a quantitative increase of more than 5% of total carotenoids. In the case of fls2 and efr-2 mutants, the carotenoid content was increased by 10% when challenged with heterologous elicitors (FIG. 10b). The results are supported by increased expression of genes related to carotenoid biosynthesis at early time points of elicitor treatment (FIGS. 11a,b).

Specific peaks that exhibit statistically significant changes for a particular elicitor was also observed. Only flg22 treatment in WT showed a statistically significant change in peak intensity at 789.7 cm⁻¹. In the literature, this vibrational band region is typically associated with chlorophyll a and phosphodiester bonds (C₅′—O—P—O—C₃′) in DNA. elf18 treatment specifically resulted in changes for the Raman peak at 1065 cm⁻¹. This band is attributed to skeletal —C—C— stretching of lipids or C—O stretch: of cell wall polysaccharides (Movasaghi et. al. 2007).

The spread in Raman peak intensities that exhibited changes was further analysed using histogram plots. It provides better classification inference with high statistical significance. It was observed that Raman shifts at 1521 cm⁻¹ and the region between 1549 and 1552 cm⁻¹ which represent carotenoids and proteins (tyrosine and tryptophan) respectively, provides the best classification between mock and treated plants (FIG. 12). As the other two carotenoid Raman peaks (1,001, 1,151) are present in lower wavenumber regions where there is significant spectral overlap from other biomolecules such as lipids and proteins, clear differentiation was not observed at these Raman peaks. The changes observed at 1521 and between 1549 and 1552 cm⁻¹ are potential Raman spectral biomarkers indicating a dynamic early PTI response in plants.

### Example 7

### Raman Spectral Analysis of Pathogen Infected Plants and Measuring the Infection Response Factor (IRF)

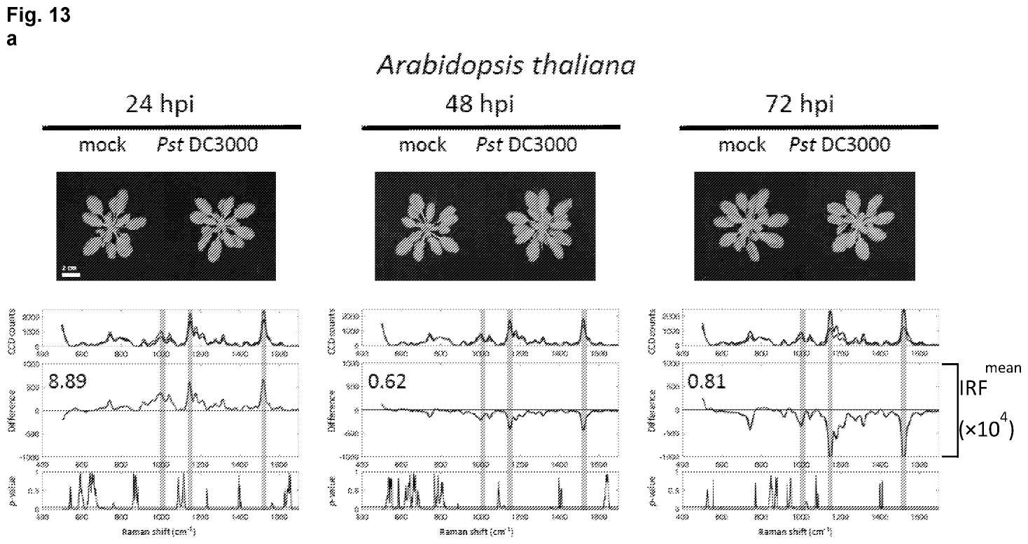

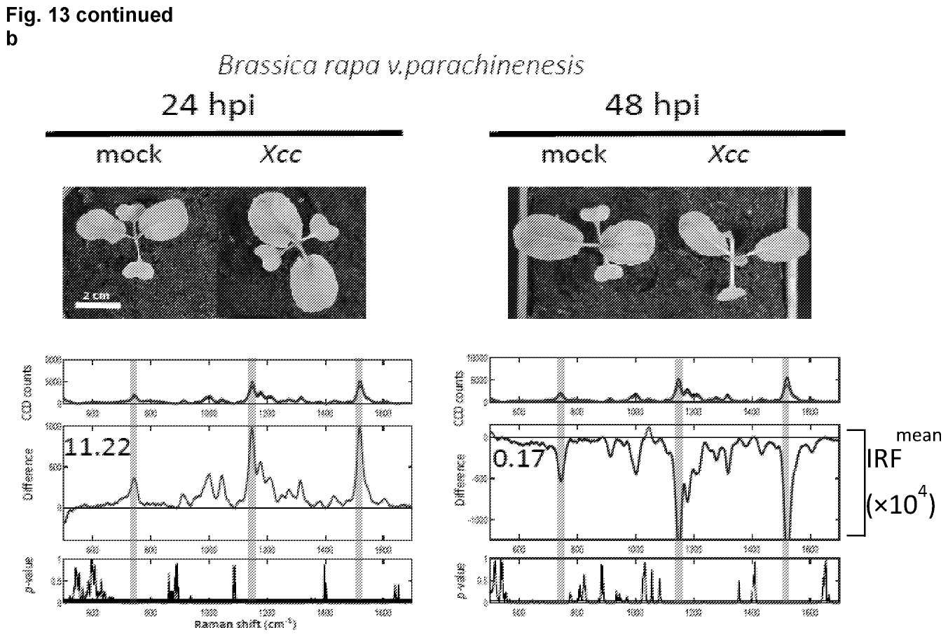

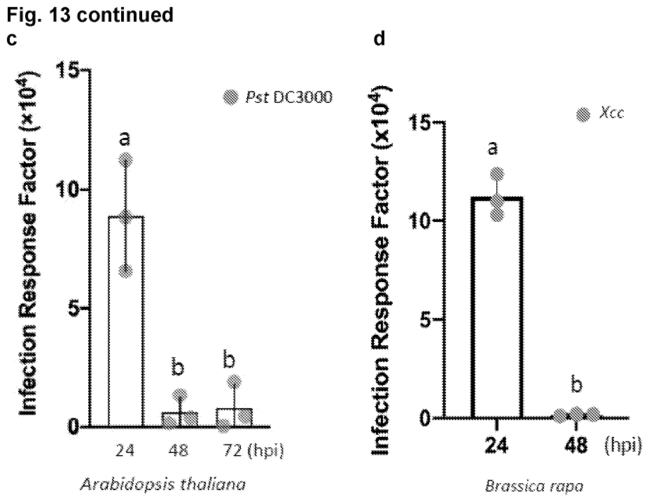

Pseudomonas syringae causes bacterial specks on Arabidopsis and induces chlorosis at late stages of infection (Whalen et al., 1991; Uppapapati et al., 2010; Luo et al., 2017). To see if Raman spectroscopy can be used to detect early Pseudomonas infection, we performed similar experiments in Arabidopsis infected with Pst DC3000. In these experiments, we estimated the IRF by the same method as the ERF value. Similar to the elicitor study, the difference Raman spectrum between the infected and mock sample at 24 hr hpi exhibited high IRF values. The IRF value decreased to almost zero at 48 and 72 hpi (FIGS. 13a,c).

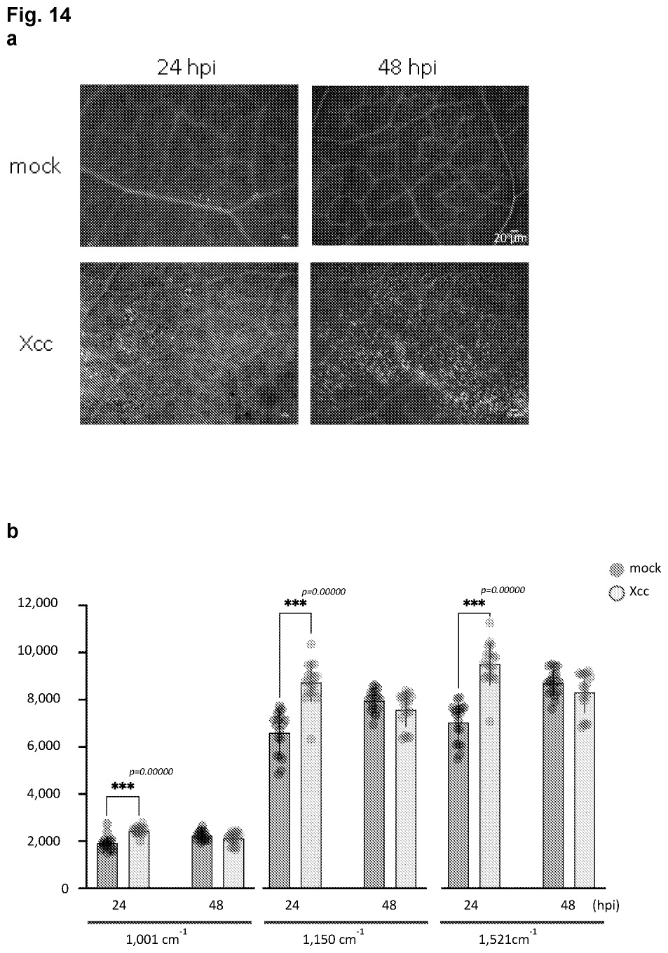

To further validate the results observed in Arabidopsis in a crop plant, Xcc infection in Choysum was analyzed. Xcc causes Black rot disease which is a serious problem in all Brassica vegetables resulting in significant yield losses. Typical external visible symptoms of Xcc infections are the chlorosis and V-shaped lesions in the leaves (Meenu et al., 2013; Nunez et al., 2018). Under our growth conditions the characteristic visual symptoms of Xcc infection are seen within 4 to 5 days. Raman spectra were measured at 24 and 48 hpi from mock and Xcc infiltrated plants to detect spectral changes before the appearance of symptoms. Like Arabidopsis, the difference Raman spectral analysis at 24 hr hpi exhibited high IRF values which declines at 48 hr (FIG. 13b,d). Similar to the Arabidopsis elicitor experiments, callose deposition was also observed in the Xcc infected leaves at 24 hpi (FIGS. 14a,b)

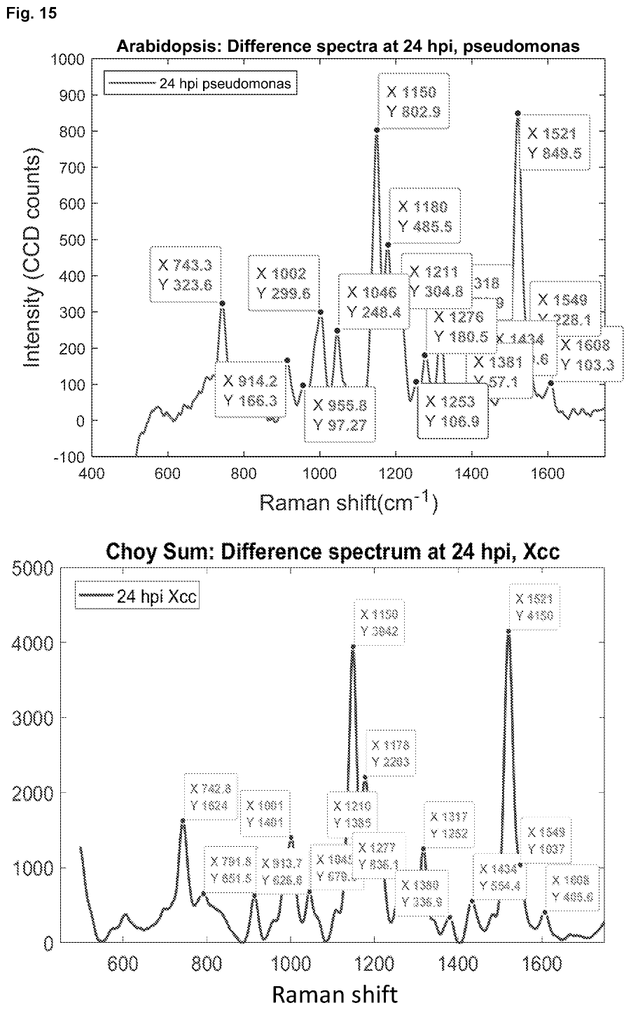

The vibrational bands contributing towards the high IRF value in Arabidopsis and Choysum were largely similar and resembled the ERF spectral data (Tables 4 and 5, FIG. 15). Like elicitor treatment, the most dominant changes upon bacterial infection in Arabidopsis and Choysum are observed around 1,001, 1,151 and 1,521 cm⁻¹ which is attributed to carotenoids (FIG. 14b) Apart from carotenoids, increase in peak intensity at vibrational bands originating from pectin, phenylpropanoids, cellulose, nitrates, nucleic acids and proteins are the dominant contributors towards the high IRF value. A significant change at 1253 cm⁻¹ peak intensity was seen only in Pseudomonas syringae infected Arabidopsis which is associated with proteins and lipids (C—N in plane stretching) while Xcc infected Choysum showed changes at 791.8 cm⁻¹attributed to nucleic acid phosphodiester bond which was not observed in Arabidopsis infection As observed with elicitor study, Raman shifts at 1521 cm⁻¹ and the region between 1549 and 1552 cm⁻¹, provides the best classification between mock and infected plants.

TABLE 4
Significant Vibrational Bands and Their Assignments that Contribute to the High
ERF Value at 24 hrs Following Pseudomonas syringae Infection in Arabidopsis

| Raman peak | | |
|---|---|---|
| (cm−1) | Assignment | Molecular bond |
| 742.3 | Pectin | γ(C—O—H) of COOH |
| | | ring br. mode (aromatics) [##] |
| 914 | Leucine, Cellulose, Lignin | (C—C), (C—N) stretching [$] |
| 956* | Carotenoids, phosphates | (C—C) [#] |
| 1001 | Phenylalanine, carotenoids | ν3 (C—CH3 stretching), |
| | | phenylalanine ring stretching mode [##] |
| 1046 | Nitrate | NO₃ stretching [##] |
| 1151 | Carotenoid | C—C stretching; v(C—O—C) [##] |
| 1180 | Carotenoid | dCH, u (pyr half-ring)ₐₛ [$$] |
| 1211 | Phenylalanine, tryptophan | u (C—C₆H₅), u₁₈ (dCₘH) [$$] |
| 1253* | Proteins, Lipids | C—N in plane stretching [#] |
| 1276 | Carotenoid | Amide III [#] |
| 1318 | Cellulose/Lignin/Protein | dCH2 bending [##] |
| 1381 | Proteins, nucleic acids | dCH3 symmetric [#] |
| 1433 | Proteins, lipids | CH₂ scissoring [#] |
| 1521 | Carotenoid | —C═C— (in-plane) [#] |
| 1549 | Amide II, Proteins: tyrosine, | Tryptophan, u (C═C) [#] |
| | tryptophan Chlorophyll a | |
| 1608 | Lignin | u (C—C) aromatic ring + s(CH) [##] |

*Denotes vibrational bands which were specific to each elicitor.
[#] Movasaghi, Zanyar, Rehman, Shazza and Rehman, Ihtesham U. (2007), Applied Spectroscopy Reviews, 42: 5, 493-541
[##] Shilpi Gupta, Chung Hao Huang, Gajendra Pratap Singh, Bongsoo Park, Nam Hai Chua, Rajeev Ram. (2020), Scientific Reports, 10, 20206.
[$] Birech Z, Mwangi P W, Bukachi F, Mandela K M (2017). PLOS ONE 12(9): e0185130
[$$] Perez-Guaita, David & Kochan, Kamila & Martin, Miguela & Andrew, Dean & Heraud, Philip & Richards, Jack & Wood, Bayden. (2016).. Vibrational Spectroscopy. 91. 10.1016/j.vibspec.2016.07.017.

TABLE 5
Significant Vibrational Bands and Their Assignments that Contribute
to the High ERF Value at 24 hrs Following Xcc Infection in Choysum

| Raman peak | | |
|---|---|---|
| (cm−1) | Assignment | Molecular bond |
| 742.8 | Pectin | γ(C—O—H) of COOH |
| | | ring br. mode (aromatics) [##] |
| 791.8* | phosphodiester | (OPO) sym [#] |
| 914 | Leucine, Cellulose, Lignin | (C—C), (C—N) stretching [$] |
| 1001 | Phenylalanine, carotenoids | ν3 (C—CH3 stretching), |
| | | phenylalanine ring stretching mode [##] |
| 1045 | Nitrate | NO₃ stretching [##] |
| 1150 | Carotenoid | C—C stretching; v(C—O—C) [##] |
| 1178 | Carotenoid | dCH, u (pyr half-ring)ₐₛ [$$] |
| 1210 | Phenylalanine, tryptophan | u (C—C₆H₅), u₁₈ (dCₘH) [$$] |
| 1277 | Carotenoid | Amide III [#] |
| 1317 | Cellulose/Lignin/Protein | dCH2 bending [##] |
| 1380 | Proteins, nucleic acids | dCH3 symmetric [#] |
| 1434 | Proteins, lipids | CH₂ scissoring [#] |
| 1521 | Carotenoid | —C═C— (in-plane) [#] |
| 1549 | Amide II, Proteins: tyrosine, | Tryptophan, u (C═C) [#] |
| | tryptophan, Chlorophyll a | |
| 1608 | Lignin | u (C—C) aromatic ring + s(CH) [##] |

*Denotes vibrational bands which were specific to each elicitor.
[#] Movasaghi, Zanyar, Rehman, Shazza and Rehman, Ihtesham U. (2007), Applied Spectroscopy Reviews, 42: 5, 493-541
[##] Shilpi Gupta, Chung Hao Huang, Gajendra Pratap Singh, Bongsoo Park, Nam Hai Chua, Rajeev Ram. (2020), Scientific Reports, 10, 20206.
[$] Birech Z, Mwangi P W, Bukachi F, Mandela K M (2017). PLoS ONE 12(9): e0185130
[$$] Perez-Guaita, David & Kochan, Kamila & Martin, Miguela & Andrew, Dean & Heraud, Philip & Richards, Jack & Wood, Bayden. (2016).. Vibrational Spectroscopy. 91. 10.1016/j.vibspec.2016.07.017.

The use of the terms “a” and “an” and “the” and similar referents in the context of describing the invention (especially in the context of any claims appended hereto) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in any claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Abdul Malik N A, Kumar I S, Nadarajah K. 2020. Elicitor and receptor molecules: Orchestrators of plant defense and immunity. International Journal of Molecular Science 21:963.
Ausubel F M. 2005. Are innate immune signaling pathways in plants and animals conserved? Nature Immunology. 6:973-979.
Baranski R, Baranska M, Schulz H. 2005. Changes in carotenoid content and distribution in living plant tissue can be observed and mapped in situ using NIR-FT-Raman spectroscopy. Planta 222, 448-457.
Beisel K G, Jahnke S, Hofmann D, Köppchen S, Schurr U, Matsubara S. 2010. Continuous turnover of carotenes and chlorophyll a in mature leaves of Arabidopsis revealed by ¹⁴CO₂ pulse-chase labeling. Plant Physiology 152:2188-2199.
Bektas Y, Eulgem T. 2015. Synthetic plant defense elicitors. Frontiers in Plant Science 5:804.
Belkhadir Y, Jaillais Y, Epple P, Balsemão-Pires E, Dangl J L, Chory J. 2012. Brassinosteroids modulate the efficiency of plant immune responses to microbe-associated molecular patterns. Proceedings of the National Academy of Sciences, USA 109:297-302.
Berens M L, Wolinska K W, Spaepen S, Ziegler J, Nobori T, Nair A, Krüler V, Winkelmüller T M, Wang Y, Mine A, Becker D, Garrido-Oter R, Schulze-Lefert P, Tsuda K. 2019. Balancing trade-offs between biotic and abiotic stress responses through leaf age-dependent variation in stress hormone cross-talk. Proceedings of the National Academy of Sciences, USA 116:2364-2373.
Berman J, Zorrilla-Lopez U, Farre G, Zhu C, Sandmann G, Twyman R M, Capell T, Christou P. 2015. Nutritionally important carotenoids as consumer products. Phytochemistry Reviews 14:727-743.
Bigeard J, Colcombet J, Hirt H. 2015. Signaling mechanisms in pattern-triggered immunity (PTI). Molecular Plant 8:521-539.
Chaudhary R, Atamian H S, Shen Z, Briggs S P, Kaloshian I. 2014. GroEL from the endosymbiont Buchnera aphidicola betrays the aphid by triggering plant defense. Proceedings of the National Academy of Sciences, USA 111:8919-8924.
Chinchilla D, Bauer Z, Regenass M, Boller T, Felix G. 2006. The Arabidopsis receptor kinase FLS2 binds flg22 and determines the specificity of flagellin perception. Plant cell 18:465-476.
Creely C M, Singh G P, Petrov D. 2005. Dual wavelength optical tweezers for confocal Raman spectroscopy. Optics Communication 245:465-470.
Daudi A, Cheng Z, O'Brien J A, Mammarella N, Khan S, Ausubel F M, Bolwell G P. 2012. The apoplastic oxidative burst peroxidase in Arabidopsis is a major component of pattern-triggered immunity. Plant Cell 24:275-287.
Dong O X, Ronald P C. 2019. Genetic engineering for disease in plants: Recent progress and future perspectives. Plant Physiology 180:26-38.
Egging V, Nguyen J, Kurouski D. 2018. Detection and identification of fungal infections in intact wheat and sorghum grain using a hand-held Raman spectrometer. Analytical Chemistry 90:8616-8621.
van Esse H P, Reuber T L, van der Does D. 2020. Genetic modification to improve disease resistance in crop. New Phytologist 225:70-86.
Fang Y, Ramasamy R P. 2015. Current and Prospective methods for plant disease detection. Biosensors 4:537-561.
Farber C, Bryan R, Paetzold L, Rush C, Kurouski D. 2020a. Non-invasive characterization of single-, double-, and triple-viral diseases of wheat with a hand-held Raman spectrometer. Frontier in Plant Science 11:01300.
Farber C, Kurouski D. 2018. Detection and identification of plant pathogens in maize kernels with a hand-held Raman spectrometer. Analytical Chemistry 90:3009-3012.
Farber C, Sanchez L, Rizevsky S, Ermolenkov A, Mccutchen B, Cason J, Simpson C, Burow M, Kurouski D. 2020b. Raman spectroscopy enables non-invasive identification of peanut genotypes and value-added traits. Scientific Reports 10:7730.
Farber C, Shires M, Ong K, Byrne D, Kurouski D. 2019. Raman spectroscopy as an early detection tool for rose rosette infection. Planta 250:1247-1254.
Farmer E E, Mousavi S, Lenglet A. 2013. Leaf numbering for experiments on long distance signaling in Arabidopsis. Protocol Exchange
Frerigmann H, Pislewska-Bednarek M, Sanchez-Vallet A, Molina A, Glawischnig E, Gigolashvili T, Bednarek P. 2016. Regulation of pathogen-triggered tryptophan metabolism in Arabidopsis thaliana by MYB transcription factors and indole glucosinolate conversion products. Molecular Plant 9:682-695.
Shilpi Gupta, Chung Hao Huang, Gajendra Pratap Singh, Bongsoo Park, Nam Hai Chua, Rajeev Ram. 2020. Portable Raman leaf-clip sensor for rapid detection of plant stress. Scientific Reports, 10, 20206.
Halter T, Imkampe J, Mazzotta S, Wierzba M, Postel S, Bücherl C, Kiefer C, Stahl M, Chinchilla D, Wang X, Nurnberger T, Zipfel C, Clouse S, Borst J W, Boeren S, de Vries S C, Tax F, Kemmerling B. 2014. The leucine-rich repeat receptor kinase BIR2 is a negative regulator of BAK1 in plant immunity. Current Biology 24:134-143.
Huang C H, Singh G P, Park S H, Chua N H, Ram R J, Park B S. 2020. Early diagnosis and management of nitrogen deficiency in plants utilizing Raman spectroscopy. Frontiers in Plant Science 11:663.
Jaillais Y, Belkhadir Y, Balsemão-Pires E, Dangl J L, Chory J. 2011. Extracellular leucine-rich repeats as a platform for receptor/coreceptor complex formation. Proceedings of the National Academy of Sciences, USA 108:8503-8507.
Jia K P, Baz L, Al-Babili S. 2017. From carotenoids to strigolactones. Journal of Experimental Botany 69:2189-2204.
Jones J D G, Dangl J L. 2006. The plant immune system. Nature 444:323-329.
Krimmer M, Farber C, Kurouski D. 2019. Rapid and noninvasive typing and assessment of nutrient content of maize kernels using a handheld Raman spectrometer. ACS Omega 4:16330-16335.
Kurenda A, Nguyen C T, Chételat A, Stolz S, Farmer E E. 2019. Insect-damaged Arabidopsis moves like wounded Mimosa pudica. Proceedings of the National Academy of Sciences, USA 116:26066-26071.
Kutschera A, Dawid C, Gisch N, Schmid C, Raasch L, Gerster T, Schäffer M, Smakowska-Luzan E, Belkhadir Y, Vlot A C, Chandler C E, Schellenberger R, Schwudke D, Ernst R K, Dorey S, Hückelhoven R, Hofmann T, Ranf S. 2019. Bacterial medium-chain 3-hydroxy fatty acid metabolites trigger immunity in Arabidopsis plants. Science 364:178-181.
Li J, Staiger C J. 2018. Understanding cytoskeletal dynamics during the plant immune response. Annual Review of Phytopathology 56:513-533.
Li X, Lu M, Tang D, Shi Y. 2015. Composition of carotenoids and flavonoids in narcissus cultivars and their relationship with flower color. Plos One 10: e0142074.
Lieber C A, Mahadevan-Jansen A. 2003. Automated method for subtraction of fluorescence from biological Raman spectra. Applied Spectroscopy 57:1363-1367.
Lopez-Molina L, Chua N H. 2000. A null mutation in a bZIP factor confers ABA-insensitivity in Arabidopsis thaliana. Plant and Cell Physiology 41:541-547.
Lopez-Molina L, Mongrand S, Chua N H. 2001. A postgermination developmental arrest checkpoint is mediated by abscisic acid and requires the ABI5 transcription factor in Arabidopsis. Proceedings of the National Academy of Sciences, USA 98:4782-4787.
Lorenz B, Wichmann C, Stöckel S, Rösch P, Popp J. 2017. Cultivation-free Raman spectroscopic investigations of bacteria. Trends in Microbiology 25:413-424.
Lu D, Lin W, Gao X, Wu S, Cheng C, Avila J, Heese A, Devarenne T P, He P, Shan L. 2011. Direct ubiquitination of pattern recognition receptor FLS2 attenuates plant innate immunity. Science 332:1439-1442.
Lu X, Tintor N, Mentzel T, Kombrink E, Boller T, Robatzek S, Schulze-Lefert P, Saijo Y. 2009. Uncoupling of sustained MAMP receptor signaling from early outputs in an Arabidopsis endoplasmic reticulum glucosidase II allele. Proceedings of the National Academy of Sciences, USA 106:22522-22527.
Luna E, Pastor V, Robert J, Flors V, Mauch-Mani B, Ton J. 2011. Callose deposition: a multifaceted plant defense response. Molecular Plant-Microbe Interactions 24:183-193.
Luo Q, Liu W W, Pan K D, Peng Y L, Fan J. 2017. Genetic interaction between Arabidopsis Qpm3.1 locus and bacterial effector gene hopW1-1 underlies natural variation in quantitative disease resistance to Pseudomonas infection. Frontiers in Plant Science
Mandrile L, Rotunno S, Miozzi L, Vaira A M, Giovannozzi A M, Rossi A M, Noris E. 2019. Nondestructive Raman spectroscopy as a tool for early detection and discrimination of the infection of tomato plants by two economically important viruses. Analytical Chemistry 91:9025-9031.
Martinelli F, Scalenghe R, Davino S, Panno S, Scuderi G, Ruisi P, Villa P, Stroppiana D, Boschetti M, Goulart L R, Davis C E, Dandekar A M. 2015. Advanced methods of plant disease detection. A review. Agronomy for Sustainable Development 35:1-25.
Meenu G, Amit V, Narender B. 2013. Black rot-A devastating disease of crucifers: A review. Agricultural Reviews 34:269-278.
Misra A K. 2014. Climate change and challenges of water and food security. International Journal of Sustainable Built Environment 3:153-165.
Movasaghi, Zanyar, Rehman, Shazza and Rehman, Ihtesham U. 2007. ‘Raman Spectroscopy of Biological Tissues’, Applied Spectroscopy Reviews, 42:5, 493-541
Nuñez AMP, Rodríguez G A A, Monteiro F P, Faria A F, Silva J C P, Monteiro A C A, Carvalho C V, Gomes L A A, Souza R M, de Souza J T, Medeiros F H V. 2018. Bio-based products control black rot (Xanthomonas campestris pv. campestris) and increase the nutraceutical and antioxidant components in kale. Scientific Reports 8:1-11.
Oerke E C. 2006. Crop losses to pests. The Journal of Agricultural Science 144:31-43.
Park B S, Yao T, Seo J S, Wong E C C, Mitsuda N, Huang C H, Chua N H. 2018. Arabidopsis NITROGEN LIMITATION ADAPTATION regulates OREI homeostasis during senescence induced by nitrogen deficiency. Nature Plants 4:898-903.
Park S H, Jeong J S, Seo J S, Park B S, Chua N H. 2019. Arabidopsis ubiquitin-specific protease UBP12 and UBP13 shape OREI levels during leaf senescence induced by nitrogen deficiency. New Phytologist 223:1447-1460.
Peng Y, van Wersch R, Zhang Y. 2018. Convergent and divergent signaling in PAMP-triggered immunity and effector-triggered immunity. Molecular Plant-Microbe Interactions 31:403-409.
Payne and Kurouski 2021 Raman-Based Diagnostics of Biotic and Abiotic Stresses in Plants. A Review. Front Plant Sci. 2021 Jan. 20; 11:616672.
Robatzek S, Chinchilla D, Boller T. 2006. Ligand-induced endocytosis of the pattern recognition receptor FLS2 in Arabidopsis. Genes & Development 20:537-542.
Rufián J S, Rueda-Blanco J, Beuzon C, Ruiz-Albert J. 2019. Protocol: an improved method to quantify activation fo systemic acquired resistance (SAR). Plant Methods 15:16.
Rys M, Juhász C, Surówka E, Janeczko A, Saja D, Tobias I, Skoczowski A, Barna B, Gullner G. 2014. Comparison of a compatible and an incompatible pepper-tobamovirus interaction by biochemical and non-invasive techniques: Chlorophyll a fluorescence, isothemal calorimetry and FTO-Raman spectroscopy. Plant Physiology and Biochemistry 83:267-278.
Saijo Y, Loo E P L, Yasuda S. 2018. Pattern recognition receptors and signaling in plant-microbe interactions. Plant Journal 93:592-613.
Sanchez L, Pant S, Xing Z, Mandadi K, Kurouski D. 2019. Rapid and noninvasive diagnostics of huanglongbing and nutrient deficits in citrus trees with a handheld Raman spectrometer. Analytical and Bioanalytical Chemistry 411:3125-3133.
Sanchez L, Ermolenkov A, Biswas S, Septiningsih E M, Kurouski D. 2020a. Raman spectroscopy enables non-invasive and confirmatory diagnostics of salinity stresses, nitrogen, phosphorus, and potassium deficiencies in rice. Frontier in Plant Science 11:573321.
Sanchez L, Ermolenkov A, Tang X T, Tamborindeguy C, Kurouski D. 2020b. Non-invasive diagnostic of Liberibacter disease on tomatoes using a hand-held Raman spectrometer. Planta 251:64.
Sanchez L, Pant S, Mandadi K, Kurouski D. 2020c. Raman spectroscopy vs quantitative polymerase chain reaction in early stage Huanglongbing Diagnostics. Scientific Reports 10:10101.
Savary S, Willocquet L, Pethybridge S J, Esker P, McRoberts N, Nelson A. 2019. The global burden of pathogens and pests on major food crops. Nature Ecology & Evolution 3:430-439.
Schneider C A, Rasband W S, Eliceiri K W. 2012. NIH Image to ImageJ: 25 years of image analysis. Nature Methods 9:671-675.
Schulz H, Baranska M, Baranski R. 2005. Potential of NIR-FT-Raman spectroscopy in natural carotenoid analysis. Biopolymers 77:212-221.
Seo J S, Diloknawarit P, Park B S, Chua N H. 2019. ELF 18-induced long noncoding RNAI evicts fibrillarin from mediator subunit to enhance pathogenesis-related gene1 (PR1) expression. New Phytologist 221:2067-2079.
Smith J M, Leslie M E, Robinson S J, Korasick D A, Zhang T, Backues S K, Cornish P V, Koo A J, Bednarek S Y, Heese A. 2014. Loss of Arabidopsis thaliana dynamic-related protein 2B reveals separation of innate immune signaling pathway. Plos Pathogens 10: e1004578.
Sng B J R, Singh G P, Van Vu K, Chua N H, Ram R J, Jang I C. 2020. Rapid metabolite response in leaf blade and petiole as a marker for shade avoidance syndrome. Plant Method 16:144.
Sumanta N, Haque C I, Nishika J, Suprakash R. 2014. Spectrophotometric analysis of chlorophylls and carotenoids from commonly growth fern species by using various extracting solvents. Research Journal of Chemical Science 4:63-69.
Sun Y, Li L, Macho A P, Han Z, Hu Z, Zipfel C, Zhou J M, Chai J. 2013. Structural basis for flg22-induced activation of the Arabidopsis FLS2-BAK1 immune complex. Science 342:624-628.
Ton J, Jakab G, Toquin V, Flors V, lavicoli A, Maeder M N, Metraux J P, Mauch-Mani B. 2005. Dissecting the β-aminobutyric acid-induced priming phenomenon in Arabidopsis. Plant Cell 17:987-999.
Uppalapati S R, Ishiga Y, Ryu C M, Ishiga T, Wang K, Noël L D, Parker J E, Mysore K S. 2010. SGT1 contributes to coronatine signaling and Pseudomonas syringae pv. tomato disease symptom development in tomato and Arabidopsis. New Phytologist
Velásquez A, Castroverde C D M, He S Y. 2018. Plant and pathogen warfare under changing climate conditions. Current Biology 28:619-634.
Wang J, Grubb L E, Wang J, Liang X, Li L, Gao C, Ma M, Feng F, Li M, Li L, Zhang X, Yu F, Xie Q, Chen S, Zipfel C, Monaghan J, Zhou J M. 2018. A regulatory module controlling homeostasis of a plant immune kinase. Molecular Cell 69:493-504.
Ward J L, Forcat S, Beckmann M, Bennett M, Miller S J, Baker J M, Hawkins N D, Vermeer C P, Lu C, Lin W, Truman W M, Beale M H, Draper J, Mansfield J W, Grant M. 2010. The metabolic transition during disease following infection of Arabidopsis thaliana by Pseudomonas syringae pv. tomato. Plant J. 2010 63 (3): 443-57.
Yeturu S, Jentzsch P V, Ciobotă V, Guerrero R, Garrido P, Ramos L A. 2016. Handheld Raman spectroscopy for the dearly detection of plant diseases: Abutilon mosaic virus infecting Abutilon Sp. Analytical Methods 8:3450-3457.
Yu X, Feng B, He P, Shan L. 2017. From chaos to harmony: Responses and signaling upon microbial pattern recognition. Annual Review of Phytopathology 55:109-137.
Zhang J, Li W, Xiang T, Liu Z, Laluk K, Ding X, Zou Y, Gao M, Zhang X, Chen S, Mengiste T, Zhang Y, Zhou J M. 2010. Receptor-like cytoplasmic kinases integrate signaling from multiple plant immune receptors and are targeted by a Pseudomonas syringe effector. Cell Host & Microbe 7:290-301.
Zhou J, Liu D, Wang P, Ma X, Lin W, Chen S, Mishev K, Lu D, Kumar R, Vanhoutte I, Meng X, He P, Russinova E, Shan L. 2018. Regulation of Arabidopsis brassinosteroid receptor BRI1 endocytosis and degradation by plant U-box PUB12/PUB13-mediated ubiquitination. Proceedings of the National Academy of Sciences, USA 115:1906-1915.
Zhou J, Lu D, Xu G, Finlayson S A, He P, Shan L. 2015. The dominant negative ARM domain uncover multiple functions of PUB13 in Arabidopsis immunity, flowering, and senescence. Journal of Experimental Botany 66:3353-3366.
Zhou M, Wang W. 2018. Recent advances in synthetic chemical inducers of plant immunity. Frontiers in Plant Science 9:1613.
Zipfel C, Kunze G, Chinchilla D, Caniard A, Jones J D G, Boller T, Felix G. 2006. Perception of the bacterial PAMP E F-Tu by the receptor EFR restricts Agrobacterium-mediated transformation. Cell 125:749-760.
Zipfel C, Robatzek S, Navarro L, Oakeley E J, Jones J D G, Felix G, Boller T. 2004. Bacterial disease resistance in Arabidopsis through flagellin perception. Nature 428:764-767.

1. A method of real time detection and quantitation of an innate immunity response in a plant, the method comprising:
(a) obtaining a Raman spectra of carotenoids and/or proteins in vivo and in situ in tissue of a plant leaf from a plant in a plant population, wherein the Raman spectra includes one or more peaks characteristic of carotenoids and/or proteins;
(b) obtaining a Raman spectra of carotenoids and/or proteins in vivo and in situ in tissue of a plant leaf from a control plant, wherein the Raman spectra includes one or more peaks characteristic of carotenoids and/or proteins;
(c) comparing intensity of the one or more peaks characteristic of carotenoids and/or proteins from the Raman spectra obtained from the plant in the plant population with intensity of the one or more peaks characteristic of carotenoids and/or proteins from the Raman spectra obtained from the control plant; and
(d) determining if there is an increase in the relative intensity of one or more peaks characteristic of carotenoids and/or proteins from the Raman spectra obtained from the plant in the plant population,
wherein an increase in relative intensity of the one or more peaks characteristic of carotenoids and/or proteins from the Raman spectra obtained from the plant in the plant population is indicative of an early stage infection.

2. The method of claim 1, wherein the tissue of the plant leaf is a leaf blade.

3. The method of claim 1, wherein the one or more peaks characteristic of carotenoids in the Raman spectra are selected from the group of peaks consisting of 1001 cm⁻¹, 1151 cm⁻¹ and 1521 cm⁻¹, and wherein the peak characteristic of proteins in the Raman spectra a peak at 1550 cm⁻¹.

4. The method of claim 1, wherein the Raman spectra is obtained using near-infrared excitation wavelength.

5. The method of claim 4, wherein the near-infrared excitation wavelength is 830 nm.

6. The method of claim 1, wherein obtaining the Raman spectra is non-invasive and non-destructive to the tissue of the plant leaf.

7. The method of claim 1, wherein the real time detection and quantitation of an innate immunity response in a plant is applied to urban farming.

8. The method of claim 1, wherein the real time detection and quantitation of an innate immunity response is applied to open farming.

9. A method of containing the spread of pathogen infection of plants in a plant population comprising:
detecting and quantifying an innate immunity response in a plant in a plant population real time according to the method of claim 1 to identify infected plants; and
removing infected plants from the plant population.

10. A method of real time detection and quantitation of an innate immunity response in a plant, the method comprising:
determining an Elicitor Response Factor (ERF) for a plant in a plant a plant population; and
correlating the ERF with a quantitative measure of the degree of pattern-triggered immunity (PTI);
wherein a high ERF value is associated with a high degree of PTI in the plant in the plant population and is indicative of an early stage infection.

11. The method of claim 10, wherein the ERF is determined by the steps of:
Step 1: obtaining the mean of 60 Raman spectra from each individual biological sample in the Raman shift spectral range of 400 cm⁻¹-1,700 cm⁻¹ after pre-processing the Raman spectra;
Step 2: deriving the difference of the mean spectra obtained in Step 1 between elicitor-treated and mock control-treated samples to highlight different Raman spectral regions with positive values;
Step 3: obtaining a -value plot using a t-test to evaluate the statistical significance of differential Raman spectra obtained in Step 2, wherein the differential Raman spectral regions where p-value <0.05 are noted and wherein the p-value plot was corrected by including an estimation of the positive false discovery rate (pFDR) and applying the multiple-hypothesis testing principle;
Step 4: defining the area under the curve of the differential Raman spectral region which has a positive value and also represents the corrected p-value <0.05 of Step 3 is the ERF, wherein the ERF measures the level of PTI response and wherein a higher ERF value indicates a higher level of the elicitor-induced immune response; and
Step 5: tabulating all the positive spectral regions contributing towards the ERF.

12. The method of claim 11, wherein the pre-processing comprises cosmic ray removal, Savitsky-Golay smoothing, and polynomial background subtraction.

13. The method of claim 11, wherein the biological sample is a sample of leaf tissue of a plant in a plant population.

14. The method of claim 13, wherein the tissue of the plant leaf is a leaf blade.

15. The method of claim 10, wherein the Raman spectra is obtained using near-infrared excitation wavelength.

16. The method of claim 15, wherein the near-infrared excitation wavelength is 830 nm.

17. The method of claim 10, wherein obtaining the Raman spectra is non-invasive and non-destructive to the tissue of the plant leaf.

18. The method of claim 10, wherein the real time detection and quantitation of an innate immunity response in a plant is applied to urban farming.

19. The method of claim 10, wherein the real time detection and quantitation of an innate immunity response is applied to open farming.

20. A method of containing the spread of pathogen infection of plants in a plant population comprising:
detecting and quantifying an innate immunity response in a plant in a plant population in real time according to the method of claim 10 to identify infected plants; and
removing infected plants from the plant population.
